(19) United States
(12) Patent Application Publication
(10) Pub. No.: US 2025/0366949 A1
(43) Pub. Date: Dec. 4, 2025

(54) TAVI Position Guidance with Real Time Fluoroscopy

(71) Applicant: St. Jude Medical, Cardiology Division, Inc., St. Paul, MN (US)

(72) Inventors: Peter J. Ness, Minneapolis, MN (US); Ajay Gopinath, Bedford, MA (US); Tyler Govek, Hopkins, MN (US)

(73) Assignee: St. Jude Medical, Cardiology Division, Inc., St. Paul, MN (US)

(21) Appl. No.: 19/200,004

(22) Filed: May 6, 2025

Related U.S. Application Data
(60) Provisional application No. 63/652,544, filed on May 28, 2024.

(51) Int. Cl. A61B 90/00; A61B 6/00; A61F 2/24

(52) U.S. Cl. CPC A61B 90/37; A61B 6/487; A61B 90/39; A61F 2/2427; A61B 2090/376

(57) ABSTRACT

A method may include implanting a prosthetic heart valve into a heart valve of the patient. The method may include generating a series of baseline fluoroscopic images of the target site under contrast encompassing at least one complete heartbeat cycle of the patient. The series of baseline fluoroscopic images may be annotated to provide an anatomical landmark annotation and/or a target annotation representing a target location for deploying the prosthetic heart valve. The prosthetic heart valve may be advanced toward the target site while mounted to or in a delivery device in a collapsed condition. Real-time fluoroscopic images of the target site may be generated while the prosthetic heart valve is located within the target site. The real-time fluoroscopic images may be displayed so that the anatomical landmark annotation and/or the target annotation is overlaid on the displayed images, and the prosthetic heart valve may be implanted.

### CROSS-REFERENCE TO RELATED APPLICATIONS

This application claims benefit to the filing date of U.S. Provisional Patent Application No. 63/652,544, filed May 28, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

According to one aspect of the disclosure, a method of implanting a medical device (e.g. a prosthetic heart valve or an occluder) into a location within a heart of the patient (e.g. a heart valve of the patient or a left atrial appendage of the patient) is provided. The location may be within a target site of the patient. The method may include generating a series of baseline fluoroscopic images of the target site of the patient while contrast media is within the target site, the series of baseline fluoroscopic images encompassing at least one complete heartbeat cycle of a heart of the patient. The series of baseline fluoroscopic images may be annotated to provide at least one of (i) an anatomical landmark annotation representing an anatomical landmark of the patient, or (ii) a target annotation representing a target location for deploying the medical device. The medical device may be advanced into the patient toward the target site while the medical device is mounted to or in a delivery device in a collapsed condition. Real-time fluoroscopic images of the target site may be generated while the prosthetic heart valve is located within the target site. The real-time fluoroscopic images may be displayed on a display device such that the (i) anatomical landmark annotation and/or the (ii) target annotation from the series of baseline fluoroscopic images is overlaid on the display of the real-time fluoroscopic images. The prosthetic heart valve may be deployed into the heart valve. Annotating the series of baseline fluoroscopic images may include providing both (i) the anatomical landmark annotation representing the anatomical landmark of the patient and (ii) the target annotation representing the target location for deploying the prosthetic heart valve. Annotating the series of baseline fluoroscopic images may include annotating each image in the series so that every image that encompasses at least one compete heartbeat cycle of the heart of the patient includes the annotation. The method may further include co-registering the series of baseline fluoroscopic images to the real-time fluoroscopic images so that each image in the series of baseline images is registered to a corresponding real-time fluoroscopic image. The co-registration may be performed so that each of the real-time fluoroscopic images that represents a given point within the complete heartbeat cycle of the heart corresponds to an image in the series of the baseline fluoroscopic images that represents the given point within the complete heartbeat cycle.

Annotating the series of baseline fluoroscopic images may include providing the anatomical landmark annotation representing the anatomical landmark of the patient, the anatomical landmark being a plane of an annulus of the heart valve. Annotating the series of baseline fluoroscopic images may include providing the target annotation representing the target location for deploying the prosthetic heart valve, the target location being a set distance from a plane of an annulus of the heart valve. The set distance may be non-zero. The set distance may result in the target annotation being positioned on an inflow side of the heart valve. The heart valve may be a native aortic valve, the prosthetic heart valve may be a prosthetic aortic valve, and the target annotation may be positioned in a ventricular side of the native aortic valve. Prior to deploying the prosthetic aortic valve into the native aortic valve, an inflow end of the prosthetic aortic valve may be aligned with the target annotation overlaid on the display of the real-time fluoroscopic images. The set distance may be based on (i) device-specific information relating to a device parameter of the prosthetic heart valve and/or (ii) patient-specific information relating to an anatomical parameter of an anatomy of the patient. During the generation of the series of baseline fluoroscopic images of the target site of the patient, at least a portion of an accessory wire may be located within the target site. The accessory wire may include a plurality of radiopaque markers, each adjacent pair of radiopaque markers spaced apart from each other along the accessory wire at a known distance. The method may also include annotating the series of baseline fluoroscopic images to draw a line between two of the radiopaque markers on the display device to correlate the known distance to a pixel size on the display device.

Generating the series of baseline fluoroscopic images of the target site of the patient may be performed with a static fluoroscopic imager. Generating the series of baseline fluoroscopic images of the target site of the patient may be performed with a dynamic fluoroscopic imager that sweeps around a point to generate the fluoroscopic images along different imaging planes. The heart valve may be a native aortic valve, the prosthetic heart valve may be a prosthetic aortic valve, and the point may be a radial center of a native annulus of the native aortic valve. The method may include generating audible and/or tactile feedback as the prosthetic heart valve moves closer (i) the anatomical landmark annotation or (ii) the target annotation overlaid on the display of the real-time fluoroscopic images. The method may include generating visual feedback as the prosthetic heart valve moves closer (i) the anatomical landmark annotation or (ii) the target annotation overlaid on the display of the real-time fluoroscopic image, wherein the visual feedback includes a change in a displayed color of (i) the anatomical landmark annotation or (ii) the target annotation overlaid on the display of the real-time fluoroscopic image. The series of baseline fluoroscopic images may be generated at a first resolution, and the real-time fluoroscopic images may be generated at a second resolution that is different from the first resolution. The first resolution may be higher than the second resolution. The series of baseline fluoroscopic images of the target site of the patient may be generated while the prosthetic heart valve is at the target site.

According to another aspect of the disclosure, a system is for assisting an implantation of a prosthetic heart valve into a heart valve within a target site of a patient. The system may include one or more memories for storing (i) a series of baseline images of the target site and (ii) annotations of the series of baseline images that provide at least one of (A) an anatomical landmark annotation representing an anatomical landmark of the patient, or (B) a target annotation representing a target location for deploying the prosthetic heart valve. The system may include one or more processors configured to capture a plurality of real-time extravascular images of the target site while the prosthetic heart valve is positioned on a delivery device within the target site in a crimped condition. The one or more processors may be configured to correlate a selected one of the plurality of real-time extravascular images to a selected one of the series of baseline images such that the selected one of the plurality of real-time extravascular images and the selected one of the series of baseline images represent a same cycle point within a cardiac cycle of the patient. The one or more processors may also be configured to display the plurality of real-time extravascular images on a display device such that the (i) anatomical landmark annotation and/or the (ii) target annotation from the series of baseline images is overlaid on the display of corresponding ones of the plurality of real-time extravascular images. The one or more processors may be further configured to detect locations of one or more markers in the plurality of real-time extravascular images. The series of baseline images may encompass at least one complete heartbeat cycle of a heart of the patient.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and

the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is an example of a fluoroscopic image showing contrast injection within an aorta showing an aortic valve.

FIG. 13 is an example of target positions overlaid on a fluoroscopic image of an aortic valve.

FIGS. 14A-14L are an example of a sequence of fluoroscopic images during one or more heart cycles with real-time updating of target positions overlaid on the sequence of fluoroscopic images.

FIG. 15 is a highly schematic cross-section of a delivery device and implant being imaged intra-procedurally within an aorta.

FIG. 16 is a highly schematic view of a multi-plane fluoroscopic imaging configuration.

FIG. 17 is a flow chart showing example steps of an example method of deploying a prosthetic heart valve using real-time fluoroscopy with annotations overlaid on the real-time fluoroscopic images.

FIG. 18 is a block diagram that illustrates an example of a computer system upon which an example may be implemented.

FIG. 19 is an example flow chart for tracking a device through the vasculature according to an aspect of the disclosure.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g., via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

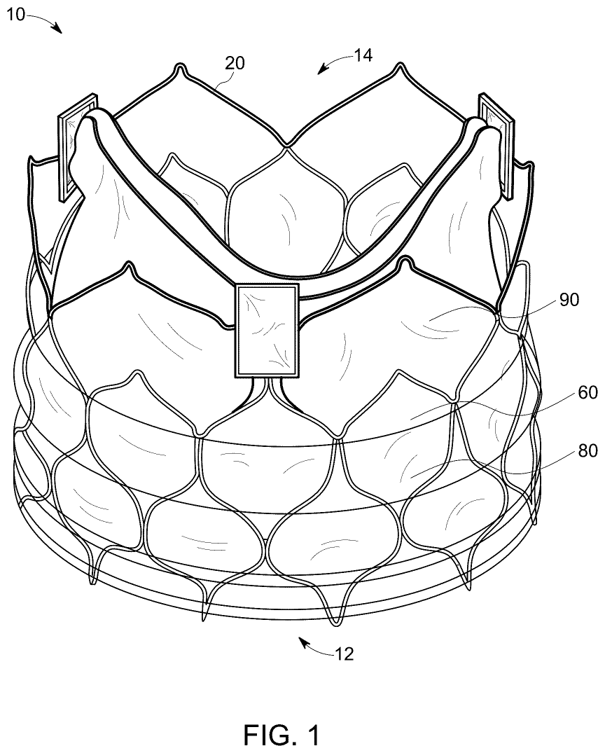

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

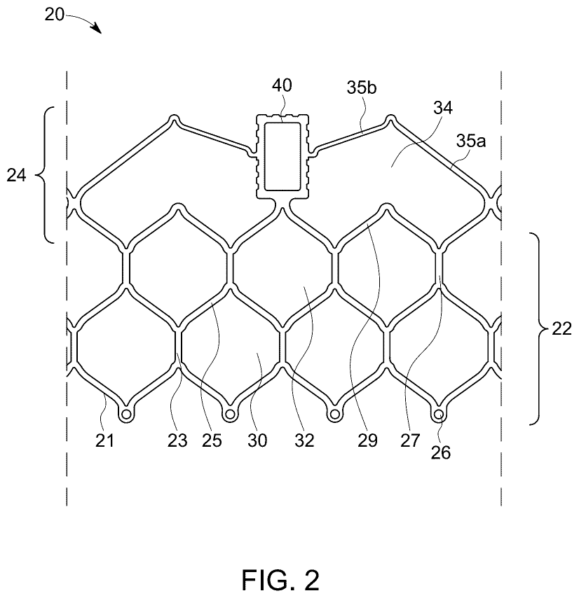

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g., where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Patent Application Publication No. 2025/0073023, the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g., coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

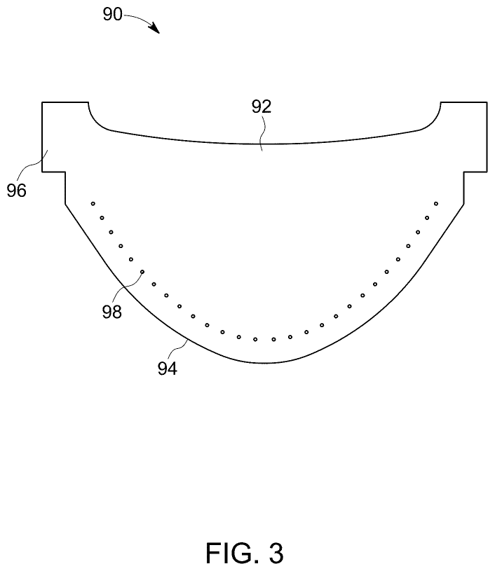

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g., via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Leaflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

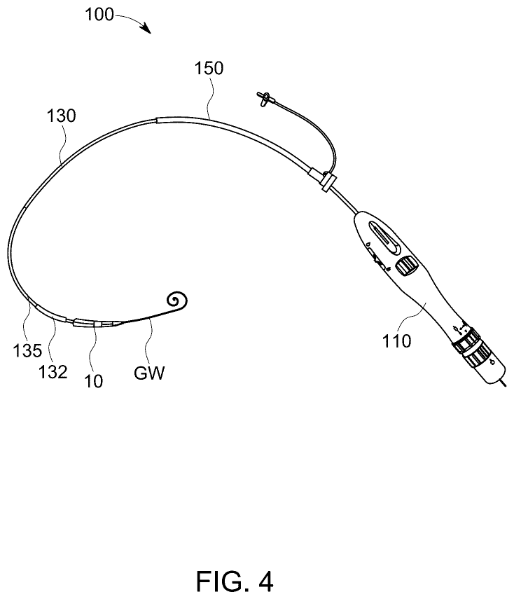

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

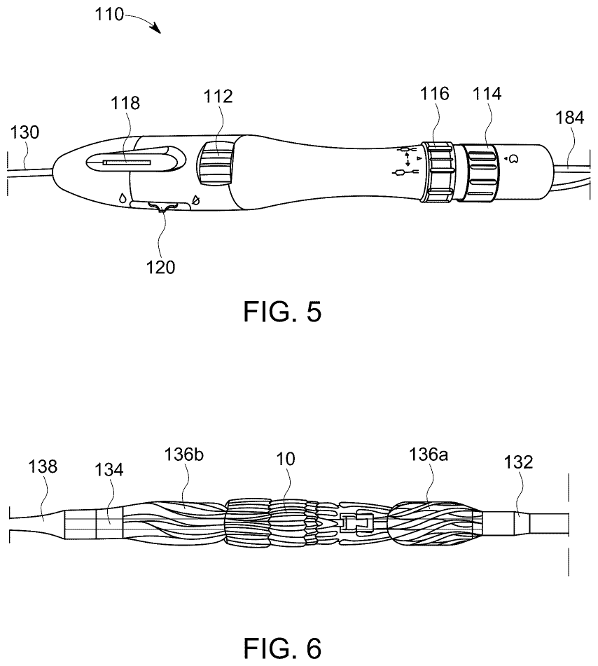

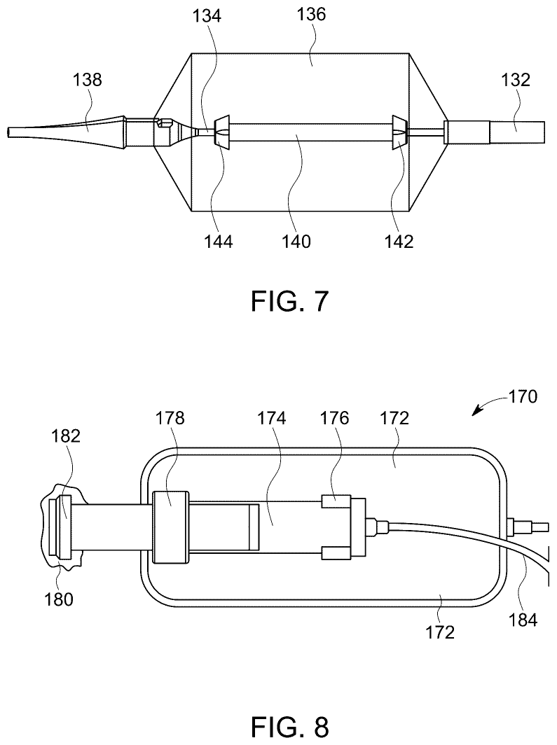

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g., within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g., within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

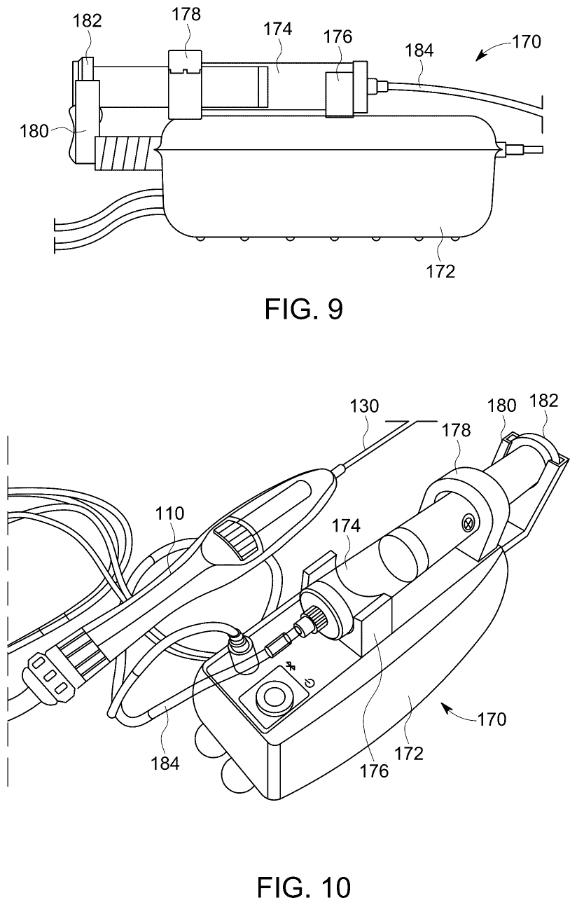

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. As used herein, the phrase “fluid reservoir” and “syringe” may be used interchangeably. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application Publication No. 2023/0372097, the disclosure of which is hereby incorporated by reference herein.

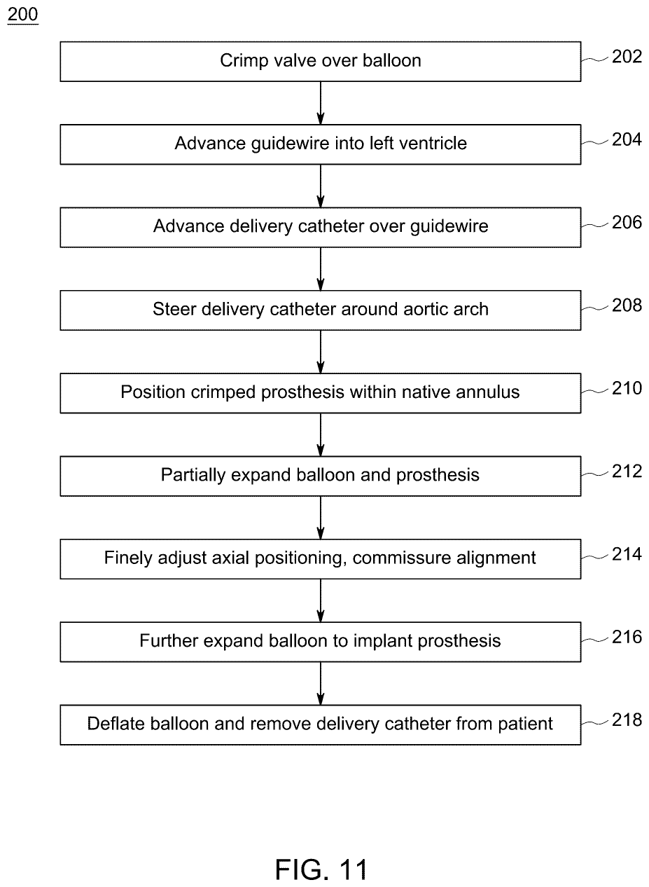

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieve and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

Typically, the success of a TAVI procedure depends, at least in part, on the accuracy of the deployment of the prosthetic heart valve within the patient's anatomy. For example, the position and orientation of the implanted prosthetic heart valve with respect to the aortic valve annulus (whether the valve annulus be a native valve annulus or previously-implanted prosthetic valve annulus), as well as the position and orientation of the implanted prosthetic heart valve relative to the left ventricular outflow tract (“LVOT”), can impact performance attributes of the implanted prosthetic heat valve, including hemodynamics, existence and/or rates of PV leak, and whether a pacemaker may need to be implanted. In some examples, extraluminal imaging, such as fluoroscopy, is performed during TAVI procedures to assist with positioning the prosthetic heart valve within the aortic valve annulus. However, fluoroscopy-which relies on x-ray images-tends to not provide sufficient resolution of soft tissue such as cardiac and/or aorta tissue for reliable determination of anatomical landmarks during the TAVI procedure. Thus, in some examples, contrast media (e.g., iodine-based contras agents such as iopamidol or iodixanol, gadolinium-based contrast agents, or other suitable contrast agents) is injected into the area of the aortic annulus, including the aorta, to better visualize the target anatomy under fluoroscopy. To further assist with navigation and/or positioning, one or more radiopaque markers or features may be provided on the delivery system and/or the prosthetic heart valve to better visualize the position of the delivery system and/or the prosthetic heart valve on the display screen. These two sources of information (e.g., anatomy visualized under contrast imaging, and delivery device (and/or prosthetic heart valve) radiopaque markers visualized under standard fluoroscopy) are sometimes used sequentially. For example, contrast injections are oftentimes brief image recordings using a high-resolution cine fluoroscopy mode. At least in part because the contrast injections are carried away with the flow of blood, there is only a limited amount of time in which the contrast media provides useful information about the surrounding anatomy before the contrast media washes out. Prosthetic heart valve and/or delivery device positioning is oftentimes performed after temporarily visualizing the target anatomy from the contrast injection using low-resolution fluoroscopy mode.

Some fluoroscopic imaging systems include memory configured to store a “roadmap” image overlay. Typically, to create a roadmap, contrast media is injected into the target anatomy (which may include the surrounding anatomy), and fluoroscopic images are captured before the contrast media washes out. The images taken under contrast may then be overlaid onto later fluoroscopic images, including those taken as the delivery device is moving through the aorta. However, this prior type of roadmapping may not provide a live image showing movement inherent in the cardiac cycle. Thus, accuracy in eventual placement of the prosthetic heart valve within the aortic valve annulus may be reduced compared to situations in which real time images and/or positioning guidance/navigation reflecting the cardiac cycle are provided.

It should be understood that the disclosure provided herein generally focuses on real-time fluoroscopic guidance in a TAVI procedure. Although some specific concepts described herein may be specific to a TAVI procedure (e.g., the use of certain anatomical landmarks relevant specifically to a TAVI procedure), it should be understood that the concepts provided herein may be readily applied to other prostheses, such as a cardiovascular stent or other transcatheter prosthetic heart valve implantation procedures, specifically including transcatheter mitral valve replacements, transcatheter pulmonary valve replacements, and transcatheter tricuspid valve replacements. Still further, the concepts provided herein may be readily applied to non-heart valve procedures, such as transcatheter left atrial appendage (“LAA”) occlusion procedures as well as other transcatheter cardiac occlusion procedures (e.g., patent foramen ovale (“PFO”) closure, atrial septal defect (“ASD”) closure, etc.).

The concepts provided herein can be applied to and/or used in conjunction with pre-TAVI intravascular imaging procedures, such intravascular imaging using an optical coherence tomography (“OCT”) probe, an intravascular ultrasound (“IVUS”) catheter, micro-OCT probe, near infrared spectroscopy (NIRS) sensor, optical frequency domain imaging (“OFDI”), or any other device that can be used to image a blood vessel. Additionally, the concepts provided herein can be applied to and/or used in conjunction with pre-TAVI intravascular data collection, such as the collection of data using a pressure wire, flow meter, or the like. According to some examples, the data received from the intravascular data collection procedure and/or intravascular imaging procedure may be used to determine and/or identify plaque burden, thin cap fibro-atheroma (“TCFA”), side branches, calcium angles, EEL detections, wall thickness, calcium detections, proximal frames, distal frames, EEL-based metrics, stent/no stent decisions, scores, recommendations for debulking and other procedures, evidence based recommendations informed by automatic detection of regions/features of interest, stent planning, etc. The determined vessel information may be co-registered with the pre-procedural images, intra-operative images, or the like. The pre-procedural images may, be, for example, fluoroscopy images captured before the TAVI procedure and intra-operative images may, for example, be fluoroscopy images captured during the procedure. In this regard, co-registering the vessel information with the pre-procedural and/or intra-operative images may allow for the vessel information to be provided for output on or relative to the pre-procedural and/or intra-operative images. The vessel information provided for output provides additional information to the user during the TAVI procedure.

In some examples, the concepts provided herein can be applied to percutaneous intervention procedures, such as stent deployment, balloon deployment, vessel prep, or the like. The percutaneous intervention procedure may occur before and/or after the TAVI procedure. In this regard, the pre-procedural images and the intra-operative images may be used to guide the percutaneous intervention device. Collectively, the intravascular imaging procedure, intravascular data collection, and percutaneous intervention procedure may be referred to herein as “intravascular procedure.”

In some examples, the concepts provided herein can be applied to and/or used in conjunction with other extraluminal images, such as CT, MRI, or the like. Information derived and/or identified from the extraluminal images, such as organs, hard tissue, bone, etc., may be co-registered with the pre-procedural images, intra-operative images, vessel data, or the like. In this regard, co-registering the vessel information with the pre-procedural and/or intra-operative images may allow for the vessel information to be provided for output on or relative to the pre-procedural and/or intra-operative images.

Co-registration of the pre-procedural images, intra-operative images, intravascular data, extraluminal information, and/or extraluminal images may be done manually, automatically, using artificial intelligence (“AI”) model(s), or the like.

In some examples, the disclosure provided herein describes ways that TAVI device placement accuracy can be optimized by providing real-time positioning guidance under imaging such as fluoroscopy. For example, disclosure provided herein may entail fluoroscopy co-registration, which may include custom imaging marker algorithms, to show TAVI device placement targets in real-time that synchronize with the cardiac cycle. In some examples, one or more pre-determined target location(s), which may be derived from a prior fluoroscopy image set, is/are aligned and/or overlaid on live fluoroscopic images. In some examples, this may provide a visual target for the user (e.g., the operator of the TAVI delivery device) to position the prosthetic heart valve and/or the delivery device, which may result in more precise delivery and deployment of the prosthetic heart valve within the patient's anatomy.

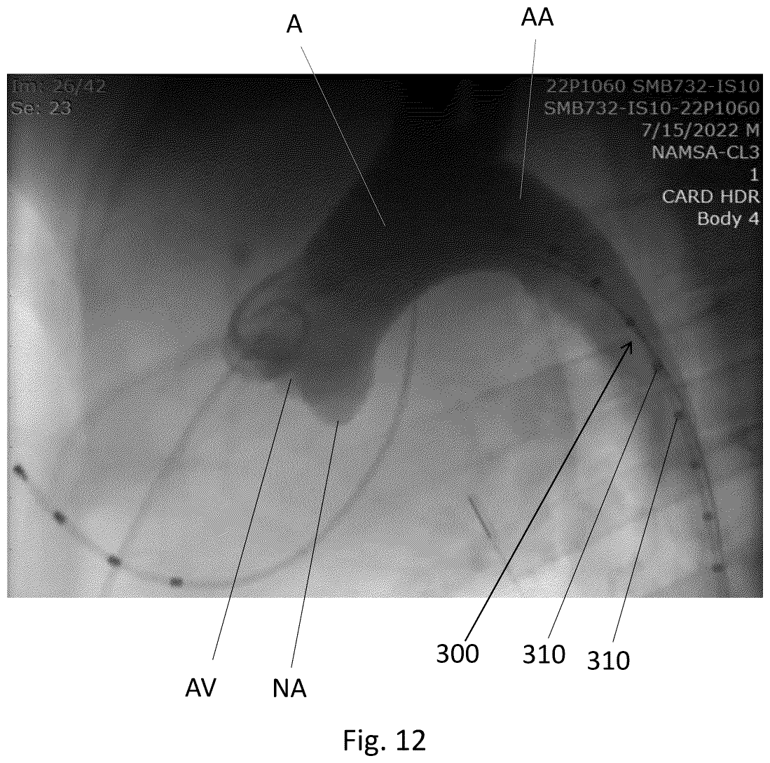

Now referring in addition to FIG. 12, FIG. 12 is an example of a fluoroscopic image (which may be displayed on a screen or other display device within the operating theater, catheter lab, or other relevant procedure site) showing contrast injection within an aorta A. In the example of FIG. 12, the contrast media helps to clearly establish the position, location and/or orientation of the native aortic valve annulus NA, the native aortic valve AV, the aorta A, and the aortic arch AA, among other anatomical landmarks. In some examples, the fluoroscopic image of FIG. 12 (or similar images taken during/following contrast injection) enables full visualization of the anatomy relevant to the TAVI procedure, and may be used as a baseline image for fluoroscopic co-registration. As explained in greater detail below, in some examples, a pigtail catheter 300 (or other wire-like accessory) having a plurality of markers 310 thereon (e.g., radiopaque markers) is also captured in the fluoroscopic image of FIG. 12. Further, it should be understood that although a single image frame is shown in FIG. 12, in some examples, a series of image frames are created similar to FIG. 12 under contrast injection throughout at least one full cardiac cycle to capture images representative of all portions of the cardiac cycle (e.g., all the events that occur from the beginning of one heartbeat to the beginning of the next heartbeat, including isovolumetric relaxation, ventricular filling, isovolumetric contraction, and ventricular ejection).

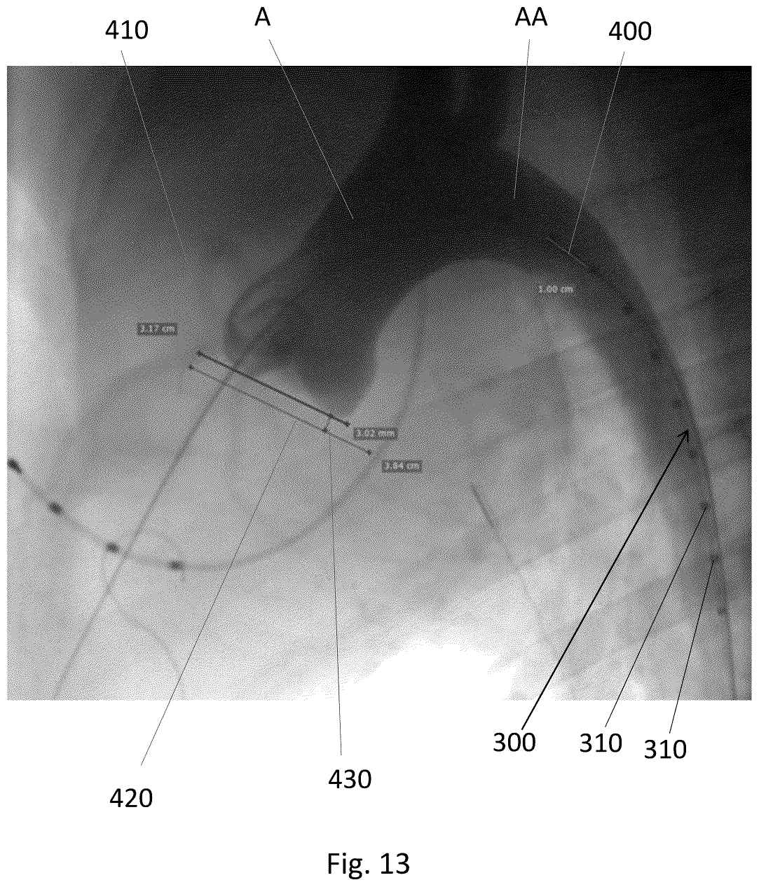

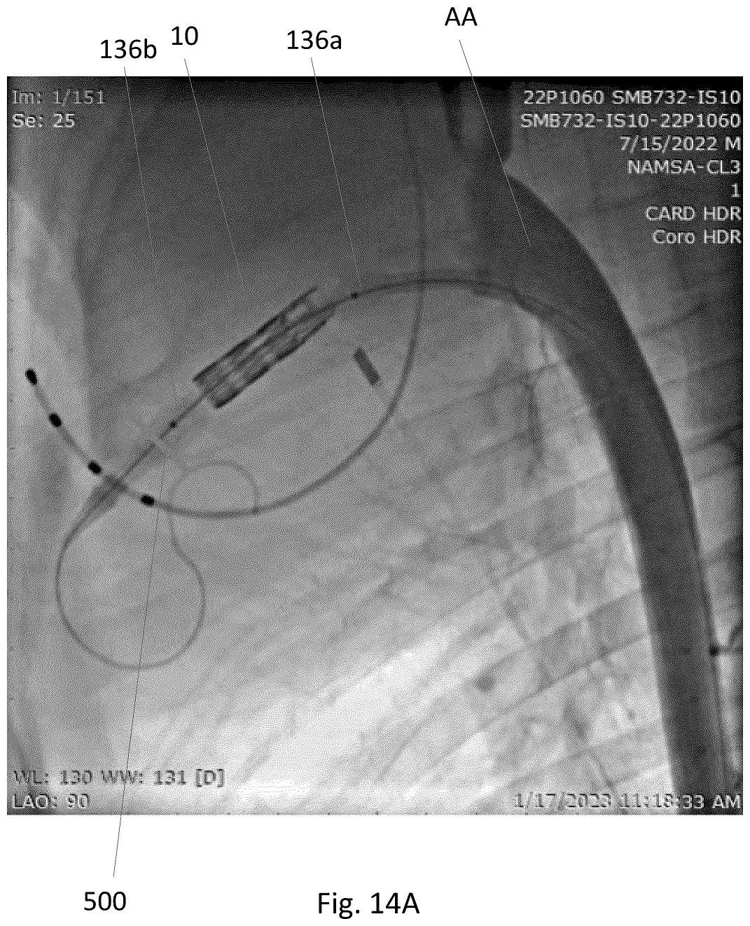

Now referring in addition to FIG. 13, FIG. 13 is an example of target positions and/or relevant information overlaid on a fluoroscopic image of an aortic valve. In some examples, as noted above, after contrast injection, a series of extraluminal images, e.g., fluoroscopic images, representing at least one full cardiac cycle are obtained. In some examples, this series of images may be analyzed, automatically, manually, or a combination thereof, to identify various relevant anatomic landmarks and parameters. For example, in the image of FIG. 13, radiopaque markers 310 on the pigtail catheter 300 been detected. In some examples, the radiopaque markers 310 may be positioned at known intervals along the length of the pigtail catheter 300, for example at 1 cm intervals. In some examples, a line 400 is drawn along the pigtail catheter 300 in the image between two adjacent markers 310. In such examples, the line 400 has a known distance equal to the known distance between adjacent markers 310 (e.g., 1 cm), and thus the line 400 may be used to determine a scale of the image shown in FIG. 13. For example, a computer system outputting the fluoroscopic image of FIG. 13 for display may correlate the number of pixels along line 400 to a distance of 1 cm to correlate pixels to actual lengths represented in the pixels. In some examples, the line 400 may be drawn manually on the image by a user providing input to the computer system, and in other examples the computer system may fully or partially autonomously determine the positions of the markers 310 along the pigtail catheter 300, for example by comparing opacities of pixels within the image and/or identifying suspected markers 310 in the image that are spaced apart at regular intervals. If this determination is being performed fully or partially autonomously, it may be performed as a background operation requiring minimal or no manual input from the user.

Still referring to FIG. 13, in some examples, in the image (or series of images) taken under contrast, anatomy that is highlighted by contrast may be detected, either automatically, manually, or a combination thereof. For example, in some examples, the outline of the aorta A may be determined in the image (or series of images) taken under contrast. In one non-limiting example, the user may manually trace the edges of the aorta A in the image (or in each image in the series of images) to allow the computer system to understand where the aorta A is positioned within the image(s). In other examples, edge detection may be used, for example by comparing opacity of pixels, in which the computer system fully or partially autonomously determines where the contours of the aorta A are positioned in the image(s). It should be understood that this type of fully or partially autonomous edge detection (under contrast) or recognition of radiopaque markers may be performed for any anatomy that is highlighted by the contrast media in the image(s), as well as for any device that includes one or more radiopaque markers that is captured in the image.

Still referring to FIG. 13, whether performed autonomously or manually, other relevant targets and/or anatomical landmarks may be annotated in the baseline image(s), preferably in each of a series of images representing at least one full cardiac cycle. In some examples, indicia 410 (e.g., a line) may be drawn or otherwise shown on the fluoroscopic images(s), with line 410 representing the plane of the native annulus NA of the aortic valve AV. In some examples, line 410 may be autonomously provided (e.g., via edge detection), manually entered (e.g., by receiving one or more user inputs corresponding to a line being drawn on the image(s) provided for output), or a combination thereof. It should be understood that target information displayed on the fluoroscopic image(s) is not limited to patient anatomy or devices within the image. For example, users may have a particular preference for where a prosthetic heart valve should be implanted relative to anatomical landmarks. In one example, a user may prefer to target aligning the inflow end of the prosthetic heart valve with a location a spaced distance (e.g., a non-zero distance, including for example 0-1 mm, 1-2 mm, 2-3 mm, etc.) from the native valve annulus VA prior to deploying (e.g., expanding) the prosthetic heart valve. In this type of example, the system may receive, as input, the preferences, which may populate another line 420 at the indicated spaced distance. In this example illustrated in FIG. 13, the spaced distance, represented by line 430, is about 3 mm. The target line 430, in some examples, is positioned at the indicated spaced distance from the native valve annulus VA represented by line 410, with line 420 being parallel to line 410. Although in some examples the system may receive an offset target distance such that the system automatically generates target line 420, in other examples, the system may receive user input corresponding to the user entering the target line 420 into the image(s), with or without assistance of the computer. In this example, during use, when the inflow end of the prosthetic heart valve is still in the collapsed condition and is aligned with target line 420, the alignment may provide an indication to the user that the prosthetic heart valve is in the desired position for deployment. Furthermore, although target line 420 is described in some examples as being based on user preference, target line 420 (or similar targets to be displayed on the fluoroscopic image(s)) may in other examples be based on a case-by-case determination of desired or optimal placement of the prosthetic heart valve (or other implantable medical device).

Although target line 420 in some examples is based on a preferred or pre-defined off-set from line 410, in some examples, target line 420 may be based on patient-specific or device-specific information. For example, different sizes or types of prosthetic heart valves may have different preferred positions relative to the plane of the valve annulus, while different anatomies of patients (e.g. the position of the coronary arteries or the size and geometry of the left ventricular outflow tract) may create different preferred positions for the prosthetic heart valve to have relative to the plane of the valve annulus. Thus, in some examples, target line 420 may be generated based on one or more of these patient-specific and/or device-specific parameters to create a personalized deployment target.

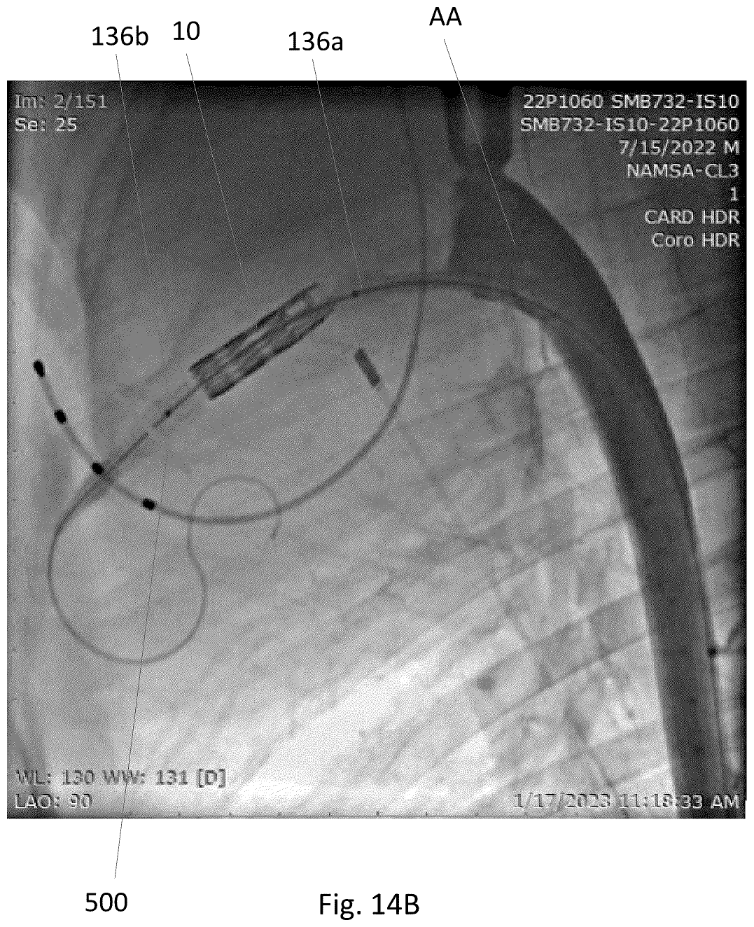

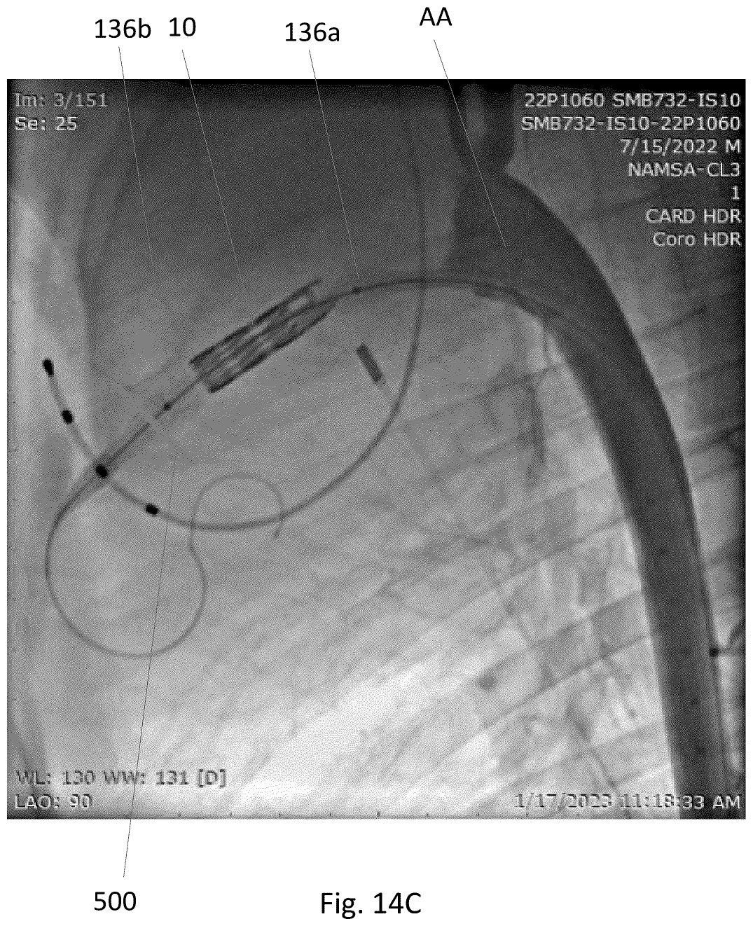

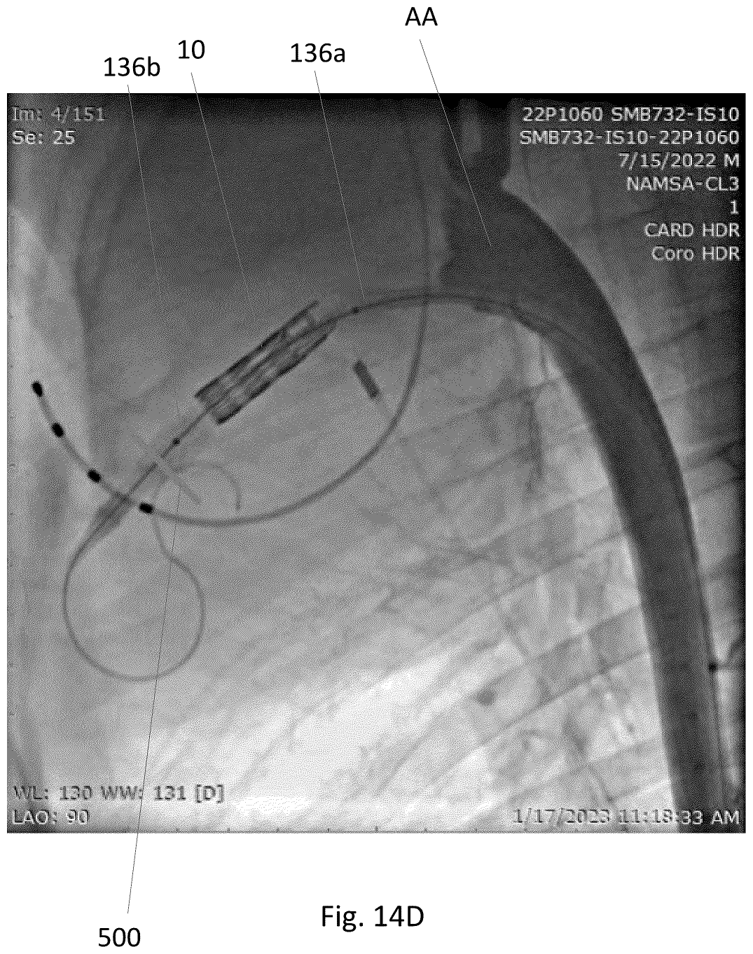

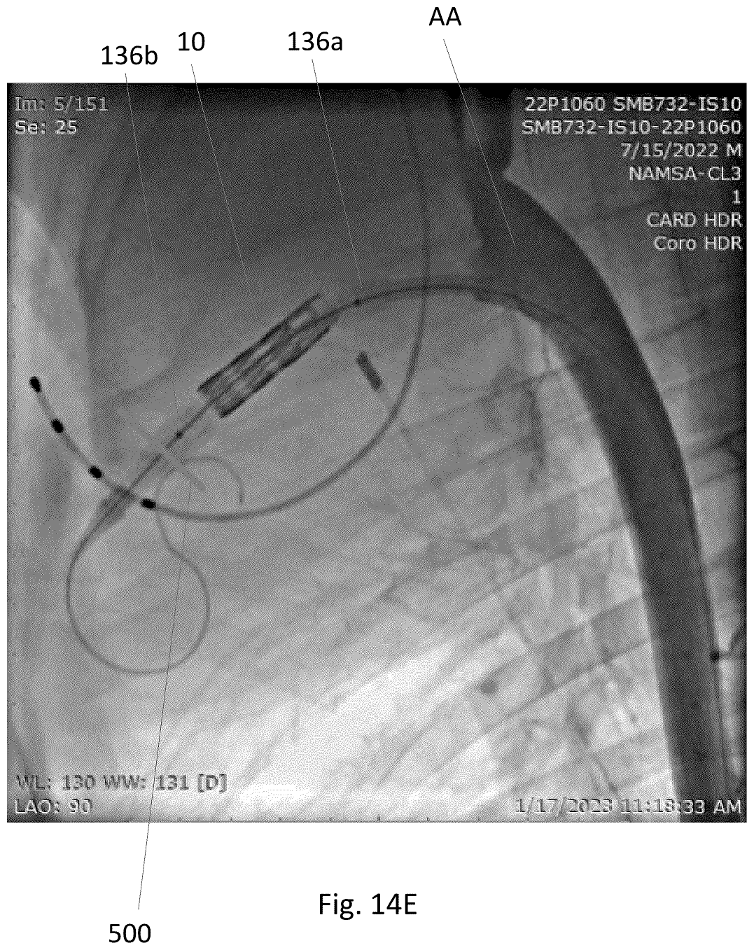

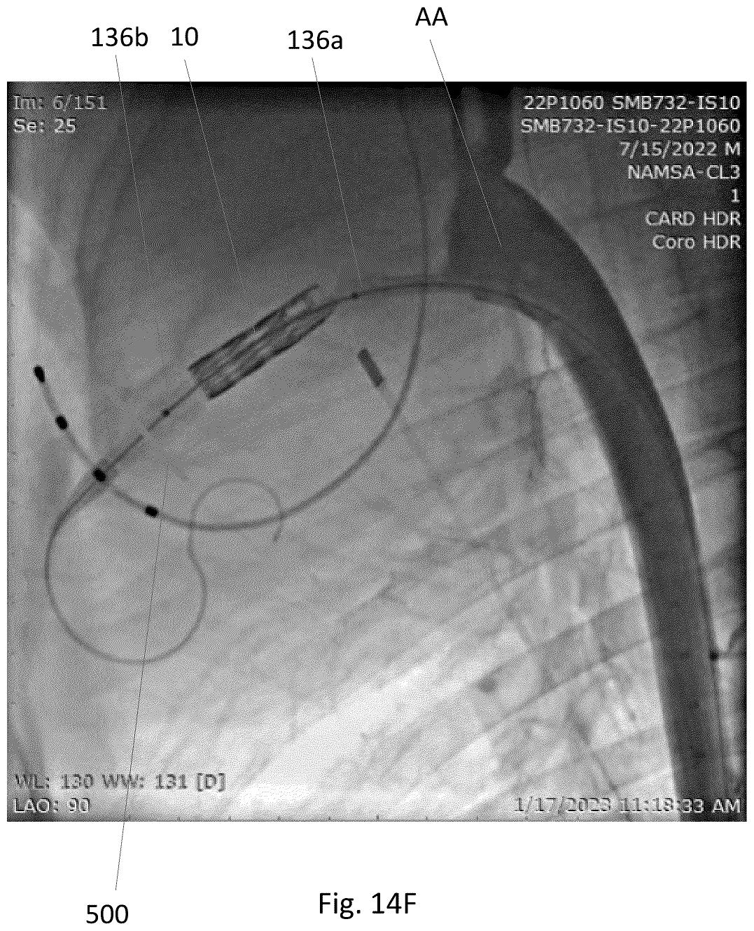

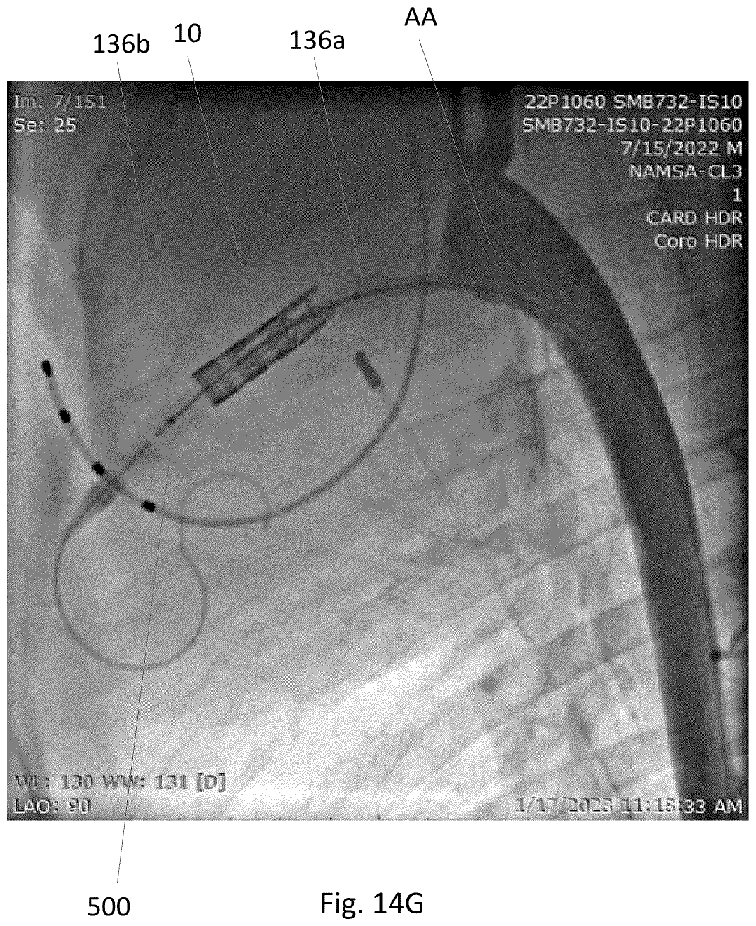

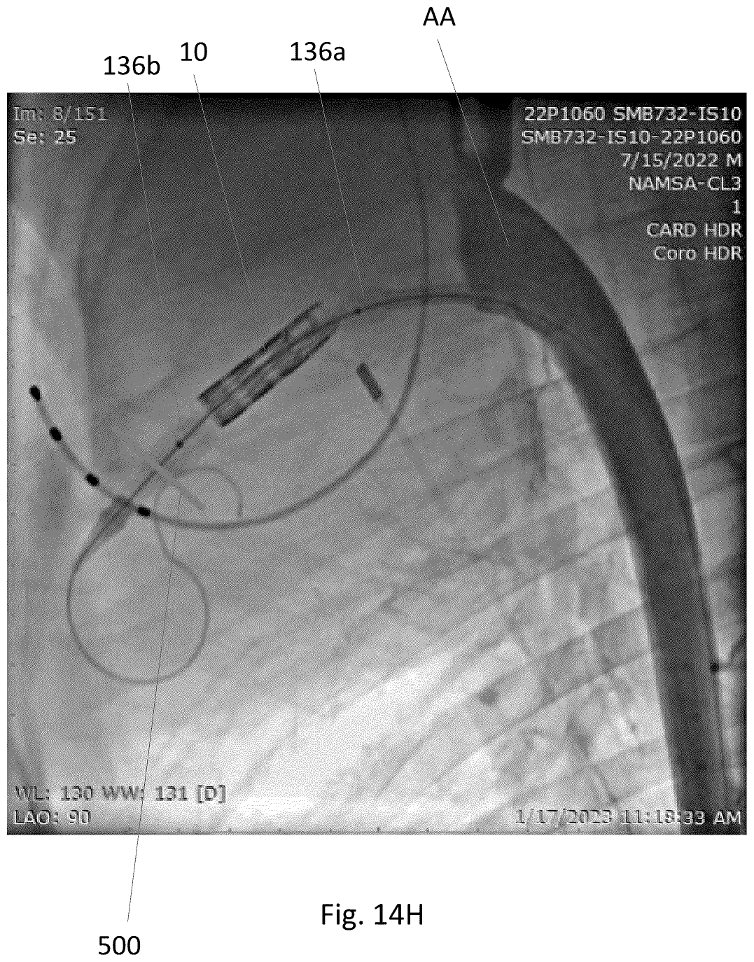

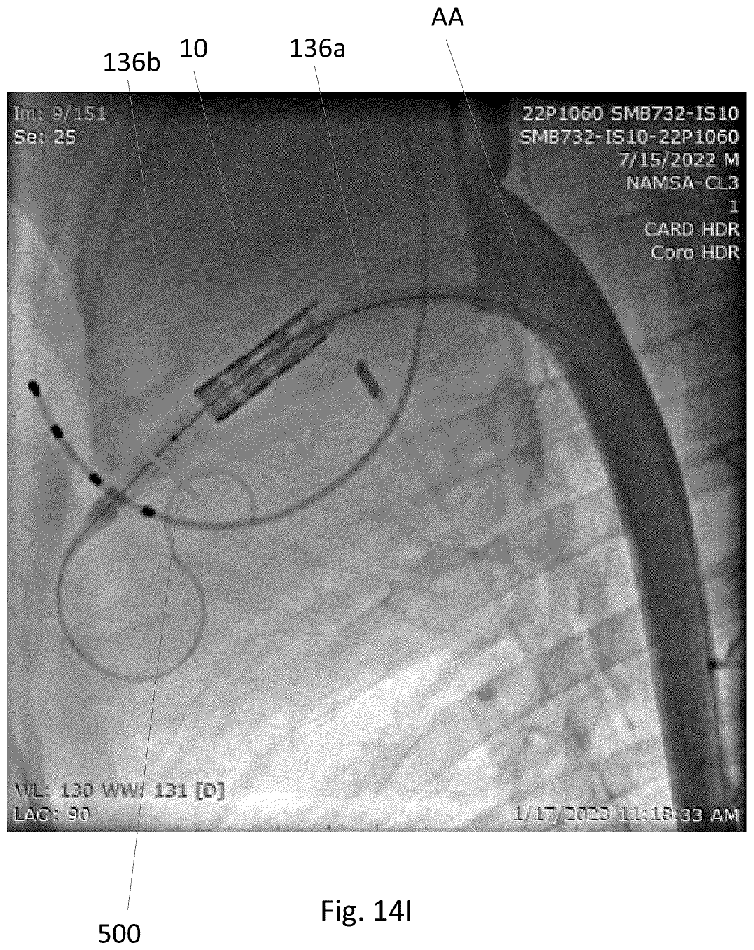

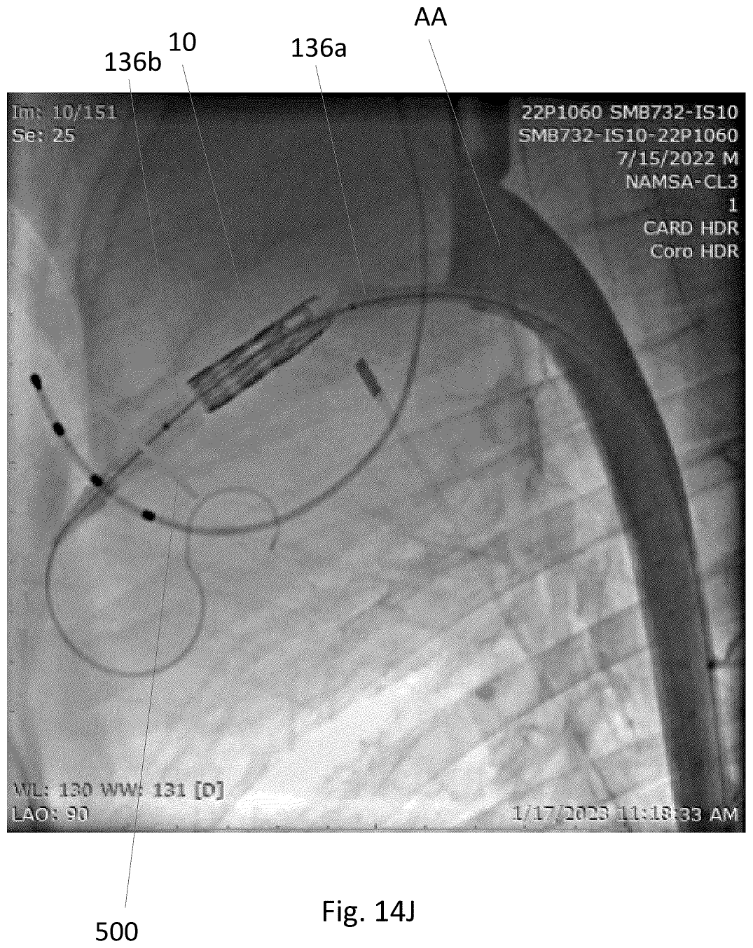

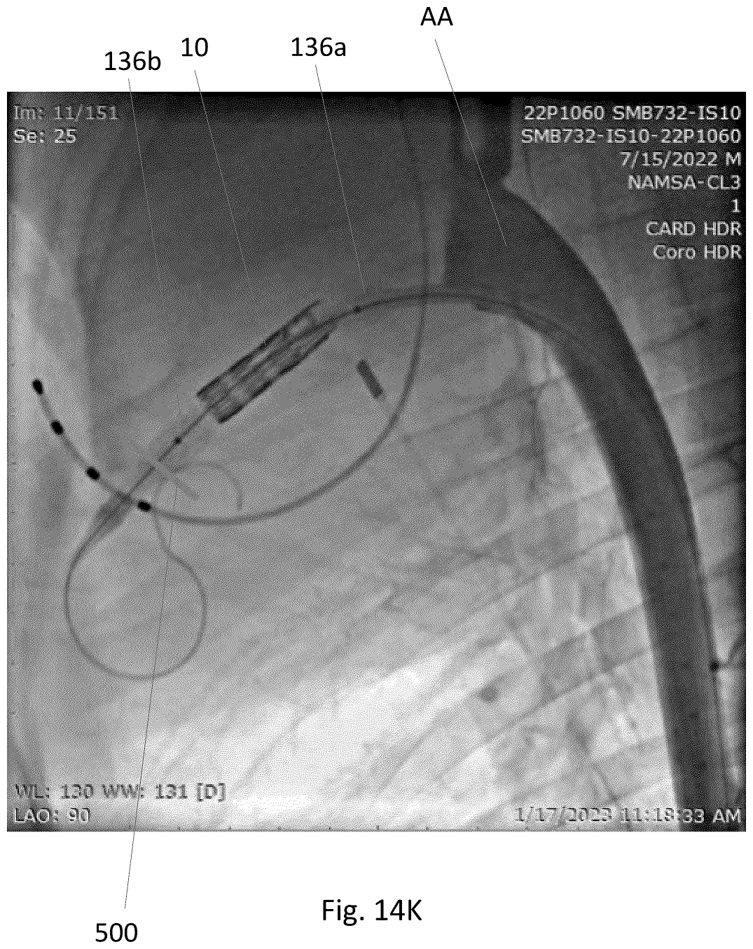

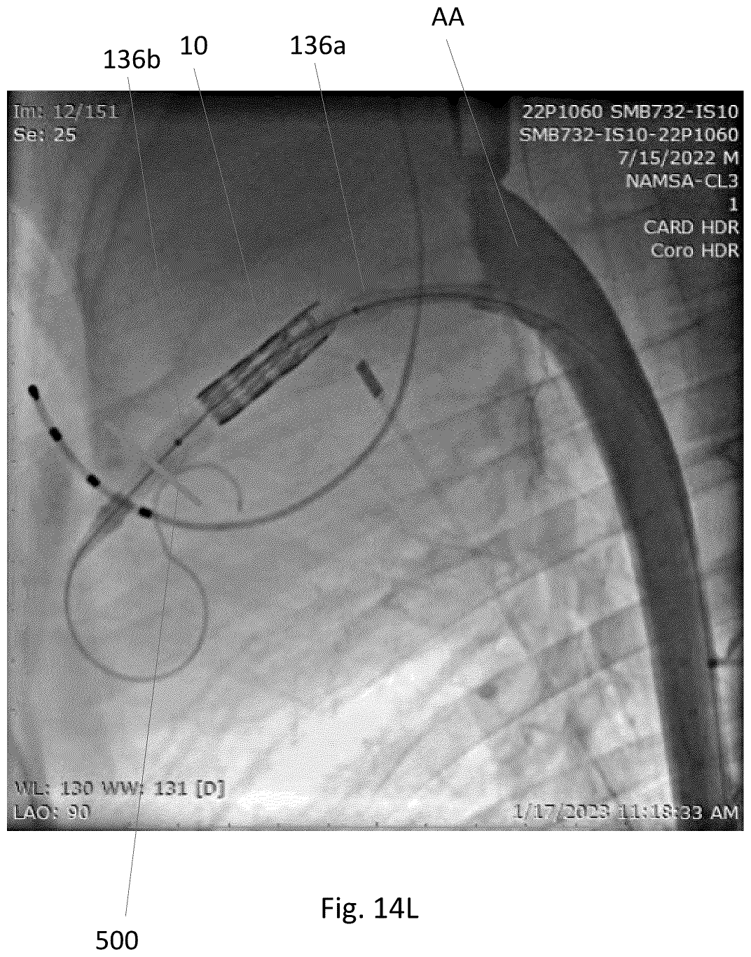

Now referring to FIGS. 14A-14L, FIGS. 14A-14L are an example of a sequence of fluoroscopic images during one or more heart cycles with real-time updating of target positions overlaid on the sequence of fluoroscopic images. In the particular example of FIGS. 14A-14L, a sequence of twelve individual fluoroscopic images in a sequence as the heart beats is provided, while prosthetic heart valve 10, which is collapsed on a delivery device between a proximal balloon pillow 136a and a distal balloon pillow 136b, is being delivered to the native aortic valve. It should be understood that, because FIGS. 14A-14L are fluoroscopic images, various components with low radiopacity are not easily discernable in the image, including for example components of the delivery device balloon. In some examples, including those shown in FIGS. 14A-14L, a radiopaque marker may be provided inside one or both of the pillows. For example, the images of FIGS. 14A-14L show one radiopaque marker easily discernable within the proximal balloon pillow 136a and another radiopaque marker easily discernable within the distal balloon pillow 136b, with a distance between the two radiopaque markers representing a working length of the balloon. Similarly, FIGS. 14A-1414L are examples of intra-procedural images in which contrast is not able to show the entirety of the target anatomy being treated, and as a result, anatomy such as the aortic valve AV and its native annulus NA may not be easily discernable on the images (compare the anatomy shown in FIGS. 12-13 to that shown in FIGS. 14A-14L).

In some examples, in order to assist the user with properly positioning the prosthetic heart valve 10, a target 500 may be provided on the intra-procedural fluoroscopic images in real-time. The target 500 may in some examples be based on targets generated in the series of fluoroscopic images taken under contrast and annotated, for example as described in connection with FIG. 13. For example, target 500 may be based on line 410 denoting the plane of the native annulus NA of the aortic valve AV, or based on line 420 denoting a target positioned a spaced distance from the native annulus NA of the aortic valve AV. Although one target 500 is shown in FIGS. 14A-14L, it should be understood that multiple targets and/or anatomical landmarks may be simultaneously annotated on the fluoroscopic images (e.g., as shown in FIGS. 14A-14L) being taken and displayed in real-time during the procedure being performed (e.g., a prosthetic heart valve implantation).

In some examples, in order to translate the targets and/or anatomical landmarks generated based on the contrast images prior to the beginning of the implant procedure (e.g., as shown in FIG. 13) to the live procedural fluoroscopic images generated and displayed intra-operatively (e.g., as shown in FIGS. 14A-14L), a process of co-registration may be performed. As part of the co-registration process, the pre-procedural images may be matched to the real-time intraprocedural images. The pre-procedural images may be extraluminal images taken with or without contrast prior to the procedure, e.g., TAVI procedure, intravascular imaging procedure, intravascular data collection procedure, percutaneous intervention procedure, etc. The pre-procedural contrast images may be matched to the real-time intraprocedural images based on the heart cycle, location of anatomical landmarks, timestamps, or the like. In some examples, because the pre-procedural images include at least one complete cycle of the heart, frames in the real-time intra-operative sequence of fluoroscopic images can be matched to an image in the sequence of pre-procedural contrast images taken at the same point within the cycle of the heart. For example, the system may be configured to identify, in real-time, during the capture of the real-time intra-operative fluoroscopic images, a given point of the heartbeat cycle associated with the captured frames. Based on the identified point of the heartbeat cycle, the system may be configured to identify a corresponding pre-procedural image taken at the same point of the heartbeat cycle. In some examples, a live fluoroscopic image or image frame may be compared by the computer system to each pre-procedural image within the cycle of one complete heartbeat to determine which pre-procedural image best matches the current live fluoroscopic image or image frame. Based on this comparison, the system may be able to register the current live fluoroscopic image or image frame to the corresponding pre-procedural image frame to register or anchor the current image to the pre-procedural image. In some examples, once registration is complete, the remaining pre-procedural images in the cycle may be cycled in real-time over the live fluoroscopic image in sequential order. In some examples, the registration may be performed multiple times, for example for each live fluoroscopic image or image frame, or for any desired number (e.g. every other, every third, every fourth, etc.) of the live fluoroscopic images or image frames, such that relevant data from the pre-procedural image set can be, in real-time, overlaid on the real-time fluoroscopic images or image frames as described in greater detail below. The system may extract previously identified anatomical landmarks and/or targets from the pre-procedural image frame and overlay, superimpose, etc. the anatomical landmarks and/or targets on the real-time intra-operative image being provided for output.

In some examples, the pre-procedural image frames may be co-registered with the intra-operative image frames based on EKG signals. For example, EKG signals may be captured during the capture of the pre-procedural images such that a given EKG signal is associated with a given pre-procedural image frame. The EKG signal may correspond to a given point of the heartbeat cycle. EKG signals may additionally be captured during the capture of the intra-operative image frames such that a given EKG signal is associated with a respective intra-operative image frame. The EKG signal associated with the intra-operative image frame corresponds to the point of the heartbeat cycle in which the intra-operative image frame was captured. The EKG signal of the intra-operative image frame may be compared with the EKG signals associated with the pre-procedural image frames. When the EKG signal associated with the intra-operative image frame matches at least one EKG signal associated with at least one pre-procedural image frame, the at least one pre-procedural image frame is identified as being captured at the same point of the heartbeat cycle as the intra-operative image frame. The pre-procedural image frames associated with an EKG signal matching an EKG signal associated with the intra-operative image are co-registered.

For example, during ventricular filling, one or more pre-procedural contrast images may have been created and annotated with relevant anatomical landmarks and/or targets. In this example, during the real-time fluoroscopic images taken and displayed intra-operatively, when the ventricle is filling in real-time, the relevant anatomical landmarks and/or targets generated in the corresponding pre-procedural contrast images may be displayed on the real-time fluoroscopic images. With this example, as the heart moves through iterations of the normal heart cycle during an implantation procedure, and while real-time fluoroscopy images of the heart (and any devices within the image frame) are displayed, despite substantially constant movement of the anatomy resulting from the beating of the heart, the target 500 (including any additional targets and/or anatomical landmarks being annotated or overlaid on the fluoroscopic images) may update along with movement of the anatomy. For example, if the sequence of real-time intra-operative image frames of FIGS. 14A-14L are viewed in order, similar to how they would be displayed in a real-time fluoroscopic intra-procedural image, the position and orientation of the target 500 continually updates to match the movement of the anatomy resulting from beating of the heart.

By co-registering the pre-procedural images and the real-time intra-operative images such that the anatomical landmarks and/or targets identified on the pre-procedural images are provided for output on the real-time intraoperative images, the user can dynamically clearly visualize the vessel morphology while delivering an intravascular device. In some examples, clear visualization of the vessel morphology while delivering the intravascular device is furthered by the co-registration of intravascular and/or extraluminal data with the pre-procedural images and/or real-time intra-operative images. Further, the automatic co-registration and display of information allows for efficient and almost fully automatic fusion of information between pre-procedural images, including pre-TAVI intravascular images, intravascular data, extraluminal images, and extraluminal data, and intra-operative images. For example, computational efficiency is increased as processing and network overhead is decreased as fewer user inputs may be received by the system. In particular, as the system is configured to automatically determine anatomical landmarks and/or targets and track the heartbeat cycle with respect to the captured images, the system receives fewer inputs, thereby requiring less processing power. Further, by providing the anatomical landmarks and/or targets as an overlay on the intra-operative image provided for output reduces processing power and network overhead as fewer images have to be provided for output. In particular, rather than provide both the pre-procedural image and the intra-operative image for output, only a single image has to be provided for output. Providing a single image for output, e.g., the intra-operative image with the anatomical landmarks and/or targets overlaid thereon, requires less processing power and network overhead as compared to providing multiple images for output, e.g., the pre-procedural image and the intra-operative image.

In some examples, anatomical position targets may be derived from known anatomical relationships from a separate pre-operative, non-invasive, imaging scan (e.g., a computed tomography “CT”) scan), which may then be imported into the fluoroscopy co-registration process. Such relations may include, in some examples, information obtained from the pigtail catheter 400 (or other wire-like accessory). In one example, the position and/or diameter of the patient's LVOT may be determined from a prior non-invasive scan, and imported during co-registration so that the LVOT diameter (which may be represented by a line) is overlaid on the live real-time fluoroscopy intra-operative images. In another example, the position and/or length of the patient's membranous septum (e.g., ventricular septum) may be determined from a prior non-invasive scan, and imported during co-registration so that the membranous septum (which may be represented by a line) is overlaid on the live real-time fluoroscopy intra-operative images. In other examples, any other relevant anatomy upstream or downstream of the native annulus NA may be derived from prior non-invasive images and imported into the real-time fluoroscopic imaging in a similar manner as described above in connection with LVOT diameter and/or membranous septum length.

In some examples, one or more markers (e.g., radiopaque markers) may be provided on the prosthesis being implanted (e.g., prosthetic heart valve 10) and/or on the delivery system (e.g., delivery system 100) being used to deliver the implant, an intravascular imaging device, an intravascular data collection device, a percutaneous intervention delivery device, or the like. If such markers are provided, additional information can be presented to the user to assist in determining that the prosthesis and/or device is aligned in the desired position relative to the one or more targets overlaid on the fluoroscopy images.

According to some examples, the system may be configured to co-register any of the pre-procedural images, intra-operative images, intravascular images (e.g., OCT, IVUS, NIRS, etc.), intravascular data (e.g., calcium deposits, lipid deposits, thrombus, thin capped fibroatheromas (TCFAs or “vulnerable plaques”), vessel normalization, side branch detection, lumen size values, stents), extraluminal images (e.g., non-invasive images, such as CT, MRI) and associated information (e.g., hard tissue, bone, organs) or the like. For example, the system may be configured to receive and store extraluminal image data, such as pre-operative images, intra-operative images, extraluminal images, etc. The system may be configured to receive and store extraluminal image data, intravascular image data and/or intravascular data. In some examples, the system may access a co-registration module to co-register the intravascular data, intravascular images, pre-operative images, intra-operative images, and/or extraluminal images. For example, the co-registration module may co-register the intravascular data and/or information associated with the extraluminal images (“extraluminal data”) with pre-operative images and/or intra-operative images such that additional information that may be helpful during the TAVI procedure is provided for output. In some examples, the co-registration module may co-register intravascular data, such as plaque burden, EEL measurement, lumen diameter measurements, vessel wall thickness, TCFAs, side branches, calcium deposits, EEL detections and/or metrics. TA user of the system may guide the TAVI procedure based, at least in part, on the co-registered information provided for output on the live fluoroscopy.

In one example, the co-registration module may co-register intraluminal data captured during a pullback with one or more pre-operative and/or intra-operative images. For example, the pre-operative and/or intra-operative images may be pre-processed. Various matrices such as convolution matrices, Hessians, and others can be applied on a per pixel basis to change the intensity, remove, or otherwise modify a given pre-operative and/or intra-operative images. The preprocessing stage may enhance, modify, and/or remove features of the pre-operative and/or intra-operative images to increase the accuracy, processing speed, success rate, and other properties of subsequent processing stages. A vessel centerline may be determined and/or calculated. In some examples, the vessel centerline may be superimposed or otherwise displayed relative to the pre-processed pre-operative and/or intra-operative images. According to some examples, the vessel centerline may represent a trajectory of the device, such as an intravascular device. In some examples, the centerline may be referred to as a trace. Additionally or alternatively, marker bands or radiopaque markers may be detected in the pre-operative and/or intra-operative images. According to some examples, the pre-operative and/or intra-operative images and the data received by an intravascular device may be co-registered based on the determined location of the marker bands.

According to some examples, the co-registration module may be configured to receive one or more user inputs corresponding to a manual co-registration of intravascular data, intravascular images, pre-operative images, intra-operative images, and/or extraluminal images. For example, the system may be configured to receive one or more user inputs identifying corresponding images (e.g., images taken at the same point of the heartbeat cycle), a location corresponding to identified vessel data, target zones, regions of interest, or the like.

The co-registration may include one or more artificial intelligence (“AI”) models, such as machine learning models. The AI model(s) may be trained to detect corresponding points, markers, targets, anatomical landmarks, etc. on two different image sets (collective “points”). The points detected on each image set may be used to align and/or match, e.g., co-register, the image sets. In some examples, the detected points may be used to spatially align intravascular and/or extraluminal data with the different image sets. The AI model(s) may provide, as output, a prediction of the co-registered image sets. Co-registered image sets may be images captured of the same location, at the same time, during the same point of the heartbeat cycle, of the same anatomical features, etc.

In some examples, the co-registration module may be configured to co-register image sets, intravascular data, and/or extraluminal data based on time stamps. For example, the intravascular data may be captured during a pullback of an intravascular imaging device. Extraluminal images and/or pre-procedural image frames may be captured during the pullback. A time stamp may be associated with the time the intravascular data, extraluminal images, and/or pre-procedural image frames were captured. The timestamps may be used by the co-registration module to match, e.g., co-register, the intravascular data, extraluminal images, and/or pre-procedural image.

In another example, the co-registration module may be configured to co-register image sets, intravascular data, and/or extraluminal data based on EKG signals and/or points during the cycle, as described above with respect to co-registering the pre-procedural images and intra-operative images.

The co-registered images and/or data may be provided for output. For example, intravascular data co-registered with the pre-operative images and/or intra-operative images may be provided for output during the TAVI procedure. The intravascular data may be provided on and/or relative to the intra-operative image frames provided for output. In some examples, the intravascular image data, extraluminal images, extraluminal data, pre-operative images, and/or intra-operative images may be used to generate one or more additional representations of the vasculature. The representations may be of a specific vessel, artery, or the like. In some examples, the representation may be a two-dimensional representation, a three-dimensional representation, a graphical representation, etc. Co-registering the images and/or data allows for the targets, anatomical features, intravascular data, etc. to be provided for output on or relative to one or more of the representations.

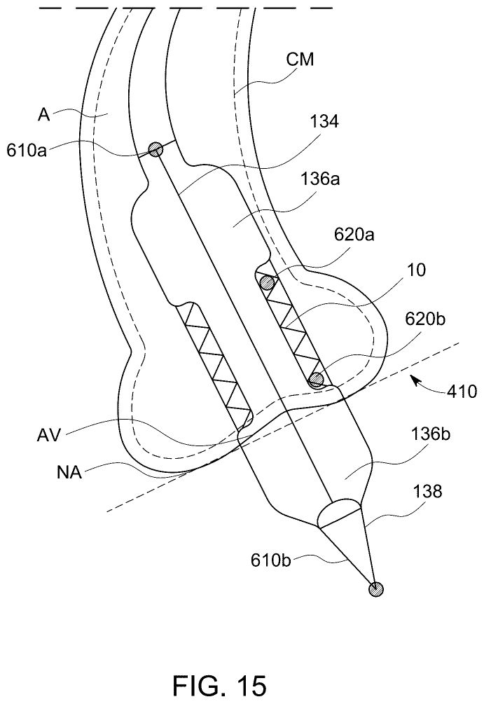

Referring now in addition to FIG. 15, FIG. 15 is a highly schematic cross-section of a delivery device (e.g., delivery system 100) and a prosthetic heart valve (e.g., prosthetic valve 10) being imaged intra-procedurally within an aorta A. In this particular example, a first marker (e.g., radiopaque marker) 610a is positioned on the delivery device, for example on inner balloon catheter 134 just proximal to proximal pillow 136a. In some examples, the first marker 610a and the inner balloon catheter 134 may extend a distance into the proximal pillow 136a. Also in this particular example, a second marker (e.g., radiopaque marker) 610b is positioned on the delivery device, for example on the atraumatic distal tip 138. In some examples, one or more markers may be provided on the prosthetic heart valve 10 itself. In the illustrated example, a first marker (e.g., radiopaque marker) 620a may be provided on the outflow end of the prosthetic heart valve 10, and a second marker (e.g., radiopaque marker) 620b may be provided on the inflow end of the prosthetic heart valve 10. In some examples, while the prosthetic heart valve 10 is crimped over the delivery device (e.g., between balloon pillows 136a, 136b) and within the patient, for example within the aorta A, contrast media CM may be injected under fluoroscopic imaging. In these examples, while under contrast, the markers on the delivery device (e.g., markers 610a, 610b) and/or on the prosthetic heart valve (e.g., markers 620a, 620b) may be determined and incorporated into the co-registration process. In other words, by registering the delivery device and/or collapsed prosthetic heart valve 10 to the real-time fluoroscopic images, the computer system may understand where the delivery device and/or prosthetic heart valve (at least while it is collapsed on or within the delivery system) is within the real-time fluoroscopic images being displayed. The system may utilize this information to provide guidance to the user regarding what delivery device and/or prosthetic heart valve features should be lined up with specific anatomical landmark(s) displayed. Referring still to FIG. 15, in one particular example, target 410 may be displayed showing the plane of the native annulus NA, similar to that shown in FIG. 13. Because the real-time fluoroscopy annotates the changing position of target 410 and is able to track the inflow end of the prosthetic heart valve 10 via marker 620b, the system may provide additional parameters and/or guidance on-screen relating to the relative position between the prosthetic heart valve 10 (and/or delivery device) versus the anatomical landmarks of interest and/or targets. For example, as the prosthetic heart valve 10 tracks around the aortic arch AA and into the aorta A, the display showing the real-time fluoroscopic images may also display how much distance remains between the inflow end of the prosthetic heart valve 10 (based on the tracked position of marker 620b) and the plane of the native annulus NA (based on the target 410 previously generated). If a different target is being used, for example target 420 shown in FIG. 13, distance between the inflow end of the prosthetic heart valve 10 and that target 420 may be displayed on screen in real-time. It should be understood that these are just two specific implementations, but various other navigation-related information could be similarly displayed. As one example, the axis of the distal end of the delivery device may be tracked based on a line passing through markers 610a, 610b, with the line between these two markers being generally coaxial with the prosthetic heart valve 10. Whether the axis of the distal end of the delivery device is coaxial with the native aortic valve AV (e.g., by determining whether the line between markers 610a, 610b is orthogonal to target 410 and/or positioned across a center of target 410) may be displayed on the screen with the real-time fluoroscopic images to help guide the user in achieving a coaxial relationship between the prosthetic heart valve 10 and the aortic valve AV. In still further examples, one or more markers (e.g., radiopaque markers) may be provided on one or more of the CAFs 40 of the frame 20. In those example, the CAFs 40 may be co-registered so that the positioning of the CAFs 40 can be tracked under real-time fluoroscopy, with the system indicating to the user whether the CAFs 40 are rotationally aligned with the commissures of the native valve leaflets.

Although some examples of providing on-screen guidance and/or navigation information helpful to the user intra-procedurally are provided immediately above, still other types of guidance and/or navigation information may be provided to the user to assist with achieving the desired or planned positioning of the prosthetic heart valve 10 within the aortic valve AV. For example, the above-described co-registration may be used to indicate when the desired device (e.g., prosthetic heart valve 10, intravascular imaging device, percutaneous intervention device, etc.) position has been achieved relative to one or more anatomical landmarks and/or annotated targets (e.g., target 410, 420). For example, when a particular device marker (e.g., marker 620b indicative of the inflow end of the prosthetic heart valve 10) converges with the desired anatomical landmark or target (e.g., annulus plane target 410 or target 420), visual and/or audible indicators may be provided to the user. For example, the targets annotated on the screen may change colors (e.g., from red to green) as the device marker converges with the annotated target. In other examples, blinking icons and/or sound effects may be provided to communicate to the user that the preferred device position, relative to the relevant anatomical landmarks and/or targets, is approaching or otherwise has been reached. In some examples, the relative positions of device markers may be pre-programmed into the system, for example with the known distance being translated into a known pixel distance on the fluoroscopic images in order to automatically identify the markers. With this information, the proximity between the relevant marker and the displayed landmark may be tracked based on a pixel distance between the marker and the landmark to track the proximity of the marker to the desired landmark. In some examples, upon detecting that the relevant marker is positioned at the relevant target, or at a desired distance relative to the relevant target (e.g., the radiopaque marker within distal pillow 136b is positioned a given distance distally to target 500), the system may initiate a signal to the balloon inflation system 170 to begin inflating the balloon 136 to deploy the prosthetic heart valve 10, with no separate user interaction (or with some desired intermediate amount of user interaction).

Although some examples of providing on-screen guidance and/or on-screen navigation information are described above, in some examples, guidance and/or navigation information feedback may additionally or alternatively be provided on or with components of the delivery system, such as delivery system 100. For example, as noted above, in some examples the handle 110 of the delivery system 100 may be operatively coupled, for example via balloon inflation system 170, to a computer display and/or computer system, which may include the computer system that is providing real-time fluoroscopic images of the implantation procedure for output. Rather than, or in addition to, providing on-screen navigation information and/or guidance, the handle 110 and/or balloon inflation system 170 may include one or more outputs, such as speakers to provide audible feedback when the device (e.g., the inflow end of the prosthetic heart valve 10) is approaching or otherwise has reached the desired position (e.g., target 410 or target 420). In some examples, the handle 110 and/or balloon inflation system 170 may be provided with a mechanism to provide tactile feedback in addition or as an alternative any of the feedback options described herein. For example, a feedback motor, which may buzz or vibrate to cause corresponding haptic feedback (e.g., buzzing or vibrating) of the handle 110 or balloon inflation system 170, may be provided within the handle 110 or the balloon inflation system 170. In these examples, the handle 110 and/or balloon inflation system 170 may provide tactile feedback to the user when the device (e.g., the inflow end of the prosthetic heart valve 10) is approaching or otherwise has reached the desired position (e.g., target 410 or target 420).

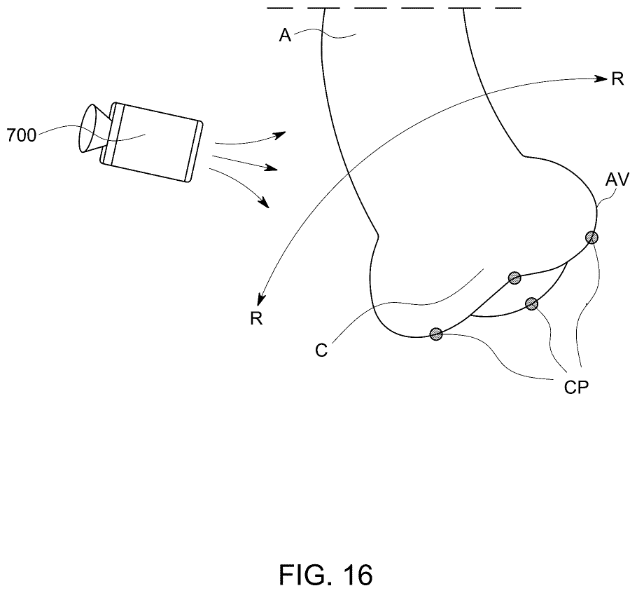

Although the real-time fluoroscopic images shown and described in connection with FIGS. 14A-14L are generally shown as single plane or two-dimensional fluoroscopic images, it should be understood that, in some examples, the concepts described herein may be applied multi-plane fluoroscopic imaging. For example, referring now in addition to FIG. 16, FIG. 16 is a highly schematic view of a multi-plane fluoroscopic imaging configuration. In the illustrated example, the patient's aorta A is shown, including the aortic valve AV. Typically, the aortic valve AV includes three native leaflets or cusps, which are shown in FIG. 16. In some examples, a center C between three cusp points CP of each leaflet may be determined, and the center C may be a point in space representing or approximating a longitudinal center of the aortic valve AV at or near the level of the plane of the native annulus NA (e.g., along target 410 shown in FIG. 15). With this center C determined, a fluoroscopic imager and/or camera 700 may be set to sweep or revolve in a direction of revolution R that is centered about the determined center C. In some examples, rather than continuous sweeping motion, multiple fluoroscopic imagers or cameras 700 may be used to create fluoroscopic images in two or more planes, which may be orthogonal planes. In either example, by taking fluoroscopic images in more than a single plane, the fluoroscopic images in different planes may be stitched together to create a three-dimensional view of the aorta A (or other anatomy relevant to the particular target site). Thus, all of the examples described herein, including using contrast images as a baseline set of images for co-registration with intra-procedural images, may be performed using a three-dimensional image set in substantially the same fashion as described herein in connection with two-dimensional image sets. According to some examples, the three-dimensional image set may be co-registered with intravascular images, intravascular data, percutaneous intervention data, or the like.

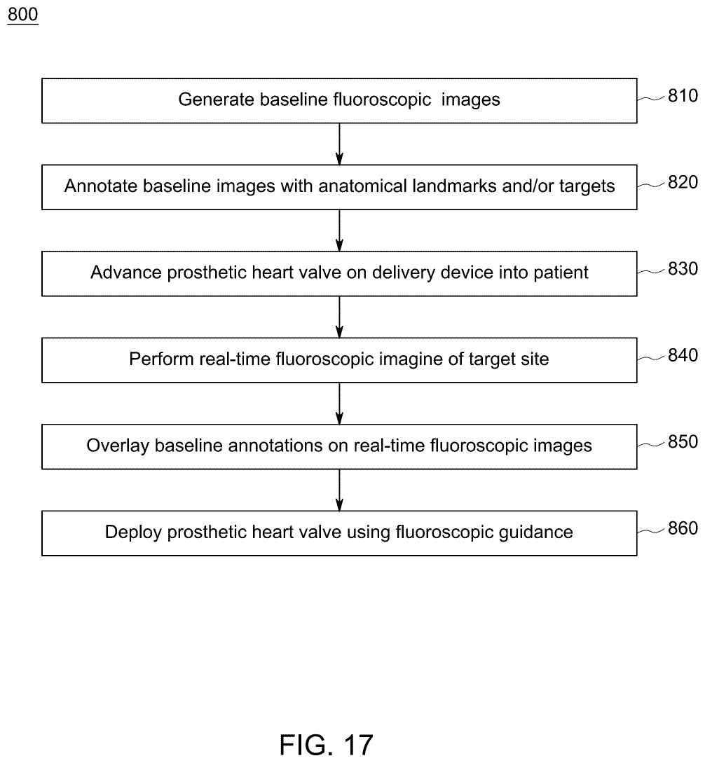

Now referring in addition to FIG. 17, FIG. 17 is a flow chart showing examples of steps of an example of a method 800 of implanting a prosthetic heart valve using real-time fluoroscopic guidance. Although method 800 is described in the context of a transcatheter aortic heart valve replacement using a balloon-expandable prosthetic heart valve, it should be understood that the method may be applicable (with appropriate step substitutions where suitable) to transcatheter aortic heart valve replacements using self-expandable prosthetic heart valves, transcatheter replacements of the mitral, pulmonary, or tricuspid valve, or other procedures such as transcatheter implantation of LAA, PFO, or ASD occluders. In some examples, in a first step 810 of method 800, a series of fluoroscopic images of the target anatomy (e.g., the aorta and surrounding anatomy including the native aortic valve) is received by the system. The series of fluoroscopic images may include at least one complete cycle of the heart beating. The images received in first step 810 may be images that were captured while or immediately after contrast media (e.g., iodine-based contrast media) is injected into the blood vessel, such as the aorta. Further, in some examples, a guidewire or other device having radiopaque markers at known spacing intervals along the device is positioned within the field of view while the fluoroscopic images are captured. The series of images may be images from a single plane, or in other examples as images in two or more planes, such as by images captured from multiple fluoroscopic imagers or cameras in different planes, or by using one or more fluoroscopic imagers or cameras that captures images while revolving around a particular point of interest, such as a center of the aortic valve or a center of the native aortic valve annulus. The series of images received in first step 810 may in some examples be referred to as baseline images. In some examples, the fluoroscopic images received in first step 810 may be high-resolution images compared to lower resolution real-time fluoroscopic images described in greater detail below.

In an example of a second step 820 of method 800, the system may annotate the baseline images received in first step 810 to provide relevant information for later use. In some examples, second step 820 may include identifying anatomical landmarks, such as the plane of the native annulus NA of the aortic valve AV (e.g., target 410 in FIGS. 13 and 15), the position and/or diameter of the LVOT, or the position and/or length of the membranous septum. In some examples of step 820, additional or alternate information may be annotated on the baseline images from step 810, including for example a target for use during implantation. In some examples, the target may be based on a physician preference, such as a target line a spaced distance from the native annulus NA of the aortic valve AV (e.g., target 420 in FIG. 13). The anatomical landmarks and/or targets may be automatically identified by the system. In some examples of step 820, device-related information may be annotated on the baseline images from step 810, including for example annotating two adjacent radiopaque markers (e.g., markers 310 in FIG. 13) positioned on a wire-like accessory (e.g., pigtail catheter 300 in FIG. 13), which may be used by the computer system to relate sizing within the fluoroscopic image(s) to actual sizing (e.g., by scaling pixel length in the image to actual length of the items shown in the image). It should be understood that, in some examples, at least some of the annotations performed in step 820 may be received as user input based on one or more inputs received in conjunction with a selection of features within series of baselines images, and drawing lines, or other annotations on the images to signify the relevant target, anatomical landmark, or other relevant information. It should be understood that, in some examples, the system may be configured to autonomously and/or automatically annotated the images. For example, the system may use edge detection algorithms to provide annotations of detected anatomical landmarks. In some examples of step 820, if the system autonomously and/or automatically annotates the series of baseline images, the annotations may be updated, corrected, or otherwise edited based on user inputs received by the system. For example, the system may receive one or more user inputs if the user finds the autonomously provided annotations incorrect or otherwise unsuitable for use as provided. In some examples, every baseline image in a sequence representing at least one full heartbeat cycle is annotated for later user.

In an example of a third step 830 of method 800, the prosthetic heart valve (e.g., prosthetic heart valve 10) may be advanced into the patient while mounted in or on a delivery device (e.g., while mounted on balloon 136 of delivery system 100). If the procedure is a transfemoral aortic valve implantation, the prosthetic heart valve may be advanced in a retrograde direction through the femoral artery, around the aortic arch AA, and into the aorta A toward the aortic valve AV.

In an example of a fourth step 840 of method 800, real-time fluoroscopic images of the target site, which may be the aorta A and/or the aortic valve AV in transcatheter aortic valve replacement procedures, may be received by the system. In some examples, the real-time fluoroscopic imaging may be performed at lower resolution than the performed in step 810. In some examples, higher resolution fluoroscopic images may be associated with higher doses of radiation compared to lower resolution fluoroscopic images. In such cases, it may be useful to perform the baseline imaging, which may require a relatively short exposure, at higher resolutions (and comparative higher but shorter radiation doses), while performing the intra-operative real-time fluoroscopy, which may require a relatively long exposure, at lower resolutions (and comparative lower but longer radiation doses). The real-time fluoroscopic images received at step 840, in some examples, may be captured at any desirable point in the process, including prior to step 830, or in other examples only when the prosthetic heart valve reaches the area of the target site. In some examples, it may be preferable to limit the overall time in which real-time fluoroscopic images are captured to reduce the overall radiation exposure required.

In an example of a fifth step 850 of method 800, which may be performed simultaneously with step 840, one or more of the annotations on the baseline images performed in step 820 may be overlaid and/or superimposed onto the real-time fluoroscopy images that are provided for output as they are received in step 840. In some examples, as the real-time fluoroscopic images are provided for output in step 850, and the anatomy shown in the displayed images changes, for example as a result of movement of the anatomy from the beating of the heart, the annotations may update to follow the movement of the anatomy. One example of this is shown and described above in connection with FIGS. 14A-14L.

According to some examples, intravascular and/or extraluminal data may be provided for output in conjunction with the real-time fluoroscopy images. For example, pre-TAVI intravascular data, identified from pre-TAVI intravascular images, and/or extraluminal may be co-registered with the baseline images and/or real-time fluoroscopy images. In such an example, the intravascular and/or extraluminal data may be provided for output on or relative to the real-time fluoroscopy images. In some examples, the intravascular and/or extraluminal data may be overlaid and/or superimposed onto the real-time fluoroscopy images that are displayed provided for output as they are generated received in step 840.

In an example of a sixth step 860 of method 800, the prosthetic heart valve may be deployed within the native valve annulus. The prosthetic heart valve may be deployed based, at least in part, on the real-time fluoroscopic images showing the position of the prosthetic heart valve relative to the one or more annotations overlaid on the real-time fluoroscopic images. In examples in which the procedure is a transcatheter aortic valve replacement using a balloon-expandable prosthetic heart valve (e.g., prosthetic heart valve 10), the real-time fluoroscopic image may be provided for output as the prosthetic heart valve is advanced (including via use of axial alignment actuator 116) until an inflow end (e.g., inflow end 12) of the prosthetic heart valve is aligned with the desired target (e.g., target 500) overlaid onto the real-time fluoroscopic images. In such an example, an indication of the prosthetic heart valve may be provided for output on the real-time fluoroscopic images in addition to any anatomical landmarks and/or targets.

According to some examples, co-registering the baseline images, intravascular data, and/or extraluminal data with the real-time fluoroscopic images based on when the images, e.g., the pre-operative and the intra-operative images, were captured allows for the anatomical landmarks and/or targets identified in the baseline images to be overlaid on the real-time fluoroscopic images. By overlaying the anatomical landmarks and/or targets from the baseline images on the real-time fluoroscopic images, the amount of contrast required during the real-time fluoroscopic imaging may be reduced. For example, by identifying a point in the heart cycle the baseline images were captured and the real-time fluoroscopic images were captured, the anatomical landmarks and/or targets identified in the baseline images may be temporally and spatially correlated such that the position of the device can be tracked in real time on the real-time fluoroscopic images relative to the vessel, anatomical landmarks, and/or targets generated from the baseline images.

In some examples, the target may be the plane of the aortic valve annulus (e.g., target 410 in FIG. 13), In another example, the target may be automatically identified based on the anatomical landmarks and/or features of the aortic valve annulus. In some examples, the target may be a target based on a predetermined criteria, including a target (e.g., target 420 in FIG. 13) that is a spaced distance from the plane of the native valve annulus. However, it should be understood that the relevant target may be any suitable target, and it should be understood that multiple targets and/or annotations may be provided for output during delivery and deployment. After the prosthetic heart valve and/or delivery device is advanced to a given position relative to the anatomy and/or target(s) overlaid on the fluoroscopic images, the prosthetic heart valve may be deployed into the native valve annulus in step 860, for example by inflating a balloon over which the prosthetic heart valve is crimped. After confirming that the prosthetic heart valve has been implanted, the delivery system may be removed from the patient, the fluoroscopic imaging may conclude, and the procedure may be completed.

It should be understood that, although the example method 800 includes example steps 810-860, more or fewer steps than those shown may be performed as part of the method, and the order of the steps may be provided in a different order than presented in the flow chart of FIG. 17 as desired by the user. It should also be understood that some or all of method 200, described in connection with FIG. 11, may be combined with some or all of method 800, as appropriate, into a single method.

As one example of an alternative or additional step in method 800, when the implant and/or delivery device (e.g., prosthetic heart valve 10 crimped on balloon 136) is at or near the target site, baseline images may be taken under contrast and annotated or otherwise co-registered to the live fluoroscopic imaging so that the live fluoroscopic imaging is able to detect landmarks on the prosthetic heart valve and/or delivery device, including for example those shown and described in connection with FIG. 15 (e.g., radiopaque markers on the inflow and/or outflow end of the prosthetic heart valve, and/or radiopaque markers on the delivery device such as the atraumatic distal tip). In some examples, the user could wait until the device is close to the target position within the annulus to generate the baseline image(s) in order to capture both the anatomy and device in the same baseline image(s).

As another example of an alternative or additional step in method 800, enhanced navigational cues may be provided for output. The enhanced navigation cues may be provided for output during the delivery and deployment of the prosthetic heart valve (e.g., as part of step 860 or prior to step 860). As used herein, the phrase “enhanced navigational cues” refers to guidance that may be provided for output beyond the overlaying of the annotations described in connection with step 850 above. For example, enhanced navigational cues may include audible feedback from the system providing the live fluoroscopic images for output. For example, the system may include one or more outputs, such as a display for providing the live fluoroscopic images for output and one or more speakers to provide audible outputs. The system may provide audible outputs when a particular part of the delivery device and/or implant is approaching or has reached the desired target. In some examples, in addition or as an alternative to audible feedback coming from the computer displaying the live fluoroscopic images, the system may instruct the delivery system or a component thereof to provide audible and/or tactile feedback, such as via speakers or vibrating motors provided in or on the handle (e.g., handle 110) of the delivery system. In some examples, the system may provide enhanced navigational cues on and/or relative to the live fluoroscopic imaging. The enhanced navigational cues may be color coded. For example, one or more targets turning from red to yellow as the relevant portion of the implant and/or delivery device approaches the target, and turning from yellow to green as the relevant portion of the implant and/or delivery device reaches the target.

It should be understood that, in some examples, the real-time fluoroscopic imaging provided for output, including annotations, may be updated during the delivery of the device. For example, rather than the annotations maintaining a static position, the system may update the position of the annotation and/or information associated with the annotations to match the constantly moving anatomy. Updating the annotations to correspond to the moving anatomy enhances the precision and ease of use in guiding a device to a target. In the example of an implant delivery device, having annotations of the real-time fluoroscopic images updated in real-time, as the anatomy moves, increase the accuracy of determining that the implant delivery device has reached the intended target. This reduces the need to correct the position of the device, thereby reducing the need to capture and/or process additional images. By increasing the efficacy of the procedure and reducing the need to correct the positioning of the device during the procedure, processing power and network overhead is reduced. For example, as the accuracy of the delivery of the device to the intended target is improved, fewer real-time fluoroscopic images need to be captured, received by the system, and processed, thereby reducing the processing power and network overhead. These types of benefits may be achieved in any anatomical implant delivery and deployment, but the benefits may be particularly evident in anatomies in or near the heart or other organs (e.g., lungs) that experience constant but cyclic movement.

As noted above, although the methods and devices are generally described herein in the context of implanting a collapsible and expandable prosthetic heart valve into a heart valve of a patient, substantially the same methods and devices may be applied to assisting in the implantation of a collapsible and expandable occluder into a left atrial appendage (“LAA”) of the patient. A LAA occluder may include a self-expanding frame, for example formed from a laser-cut tube of nitinol or formed by braiding together strands of nitinol wire and shape-setting (e.g. via heat treatment) the frame into a desired shape. In some examples, one or more fabric or other occluding members may be coupled to and/or positioned within the frame to assist with rapid occlusion of the LAA once the occluder is deployed into the LAA of the patient (e.g. by releasing the occluder from a catheter to allow the occluder to transition from a collapsed delivery condition not an expanded deployed condition which occupies at least part of the LAA to block off blood flow between the LAA and the left atrium). At least one example of a suitable LAA occluder is described in greater detail in U.S. Pat. No. 8,758,389, the disclosure of which is hereby incorporated by reference herein. Method 800 may be substantially the same as described above when applying to a LAA occlusion procedure, with the main difference being that the target site of interest is not a native heart valve, but rather the LAA of the patient, and the medical device being delivered is not a prosthetic heart valve, but a LAA occluder. And whereas the target provided in the prior example may be a plane of the annulus of the heart valve being replaced (or a target that is off-set from that plane), the target provided in this example may be a landing zone of the LAA (e.g. where contact is desired between the occluder and the internal wall of the LAA) or the plane or location of the ostium that leads from the left atrium into the LAA. Such targets may allow for an implant depth and/or a deployment angle to be tracked and updated in real-time.

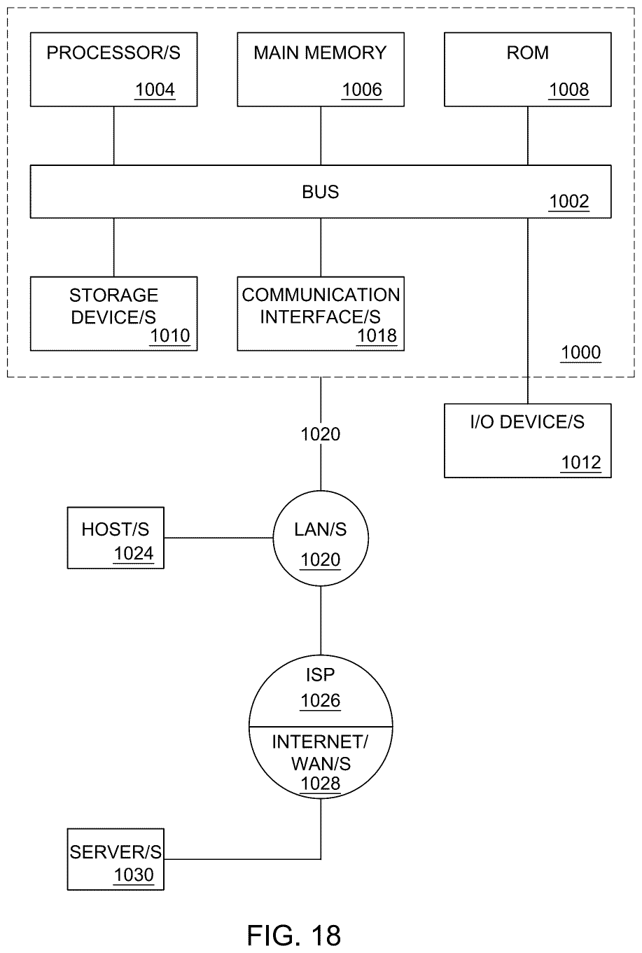

FIG. 18 is a block diagram that illustrates a computer system upon which one or more examples of the disclosure may be implemented. In some examples, processing and displaying of the fluoroscopic images described herein may be implemented on a computer system (or one or more components thereof) similar to computer system 1000 described below. In some example, at least some portions of the delivery system 1000 may interact with a computer system (or one or more components thereof) similar to computer system 1000 described below. In examples in which the handle 110 or another component of the delivery system 1000 provides feedback (e.g., audible or tactile feedback) to the user when the prosthetic heart valve 10 is approaching or has reached a target on the live fluoroscopic imaging, such feedback may be based in part via interaction between the delivery system 100 and a computer system such as computer system 1000. The computer system 1000 may include a bus 1002 or other communication mechanism for communicating information, and one or more hardware processors 1004 coupled with bus 1002 for processing information, such as computer instructions and data. The processor/s 1004 may include one or more general-purpose microprocessors, graphical processing units (GPUs), coprocessors, central processing units (CPUs), and/or other hardware processing units.

The computer system 1000 may also include one or more units of main memory 1006 coupled to the bus 1002, such as random-access memory (RAM) or other dynamic storage, for storing information and instructions to be executed by the processor/s 1004. Main memory 1006 may also be used for storing temporary variables or other intermediate information during execution of instructions to be executed by the processor/s 1004. Such instructions, when stored in non-transitory storage media accessible to the processor/s 1004, may turn the computer system 1000 into a special-purpose machine that is customized to perform the operations specified in the instructions. In some embodiments, main memory 1006 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 1000 may further include one or more units of read-only memory (ROM) 1008 or other static storage coupled to the bus 1002 for storing information and instructions for the processor/s 1004 that are either always static or static in normal operation but reprogrammable. For example, the ROM 1008 may store firmware for the computer system 1000. The ROM 1008 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 1010, such as a magnetic disk or optical disk, is provided and coupled to the bus 1002 for storing information and/or instructions. The storage device/s 1010 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media. The computer system 1000 may be coupled via the bus 1002 to one or more input/output (I/O) devices 1012. For example, the I/O device/s 1012 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 1012 may also include one or more input devices, such as an alphanumeric keyboard and/or any other keypad device. In some examples, the balloon actuators described herein may be an input device. The one or more input devices may also include one or more cursor control devices, such as a mouse, a trackball, a touch input device, or cursor direction keys for communicating direction information and command selections to the processor 1004 and for controlling cursor movement on another I/O device (e.g., a display). A cursor control device typically has degrees of freedom in two or more axes, (e.g., a first axis x, a second axis y, and optionally one or more additional axes z), that allows the device to specify positions in a plane. In some embodiments, the one or more I/O device/s 1012 may include a device with combined I/O functionality, such as a touch-enabled display.

Other I/O device/s 1012 may include a biometric sensor (e.g., fingerprint reader), a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 1004 over the bus 1002.

The computer system 1000 may implement the techniques described herein using customized hard-wired logic, one or more ASICs or FPGAs, firmware, and/or program logic which, in combination with the computer system causes or programs, causes computer system 1000 to be a special-purpose machine. In some examples, the techniques herein are performed by the computer system 1000 in response to the processor/s 1004 executing one or more sequences of one or more instructions contained in main memory 1006. Such instructions may be read into main memory 1006 from another storage medium, such as the one or more storage device/s 1010. Execution of the sequences of instructions contained in main memory 1006 causes the processor/s 1004 to perform the process steps described herein. In alternative embodiments, hard-wired circuitry may be used in place of or in combination with software instructions.

The computer system 1000 may also include one or more communication interfaces 1018 coupled to the bus 1002. The communication interface/s 1018 provide two-way data communication over one or more physical or wireless network links 1020 that are connected to a local network 1022 and/or a wide area network (WAN), such as the Internet. For example, the communication interface/s 1018 may include an integrated services digital network (ISDN) card, cable modem, satellite modem, or a modem to provide a data communication connection to a corresponding type of telephone line. Alternatively and/or in addition, the communication interface/s 1018 may include one or more of: a local area network (LAN) device that provides a data communication connection to a compatible local network 1022; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 1000 and one or more LANs 1022 and/or WANs. The network link/s 1020 typically provides data communication through one or more networks to other data devices. For example, the network link/s 1020 may provide a connection through one or more local area networks 1022 (LANs) to one or more host computers 1024 or to data equipment operated by an Internet Service Provider (ISP) 1026. The ISP 1026 provides connectivity to one or more wide area networks 1028, such as the Internet. The LAN/s 1022 and WAN/s 1028 use electrical, electromagnetic, or optical signals that carry digital data streams. The signals through the various networks and the signals on the network link/s 1020 and through the communication interface/s 1018 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 1002. Transmission media can also take the form of acoustic or light waves, such as those generated during radio-wave and infra-red data communications.

Various forms of media may be involved in carrying one or more sequences of one or more instructions to the processor 1004 for execution. For example, the instructions may initially be carried on a magnetic disk or solid-state drive of a remote computer. The remote computer can load the instructions into its main memory 1006 and send the instructions over a telecommunications line using a modem. A modem local to the computer system 1000 can receive the data on the telephone line and use an infra-red transmitter to convert the data to an infra-red signal. An infra-red detector can receive the data carried in the infra-red signal and appropriate circuitry can place the data on the bus 1002. The bus 1002 carries the data to main memory 1006, from which the processor 1004 retrieves and executes the instructions. The instructions received by main memory 1006 may optionally be stored on the storage device 1010 either before or after execution by the processor 1004.

The computer system 1000 can send messages and receive data, including program code, through the network(s), the network link 1020, and the communication interface/s 1018. In the Internet example, one or more servers 1030 may transmit signals corresponding to data or instructions requested for an application program executed by the computer system 1000 through the Internet 1028, ISP 1026, local network 1022 and a communication interface 1018. The received signals may include instructions and/or information for execution and/or processing by the processor/s 1004. The processor/s 1004 may execute and/or process the instructions and/or information upon receiving the signals by accessing main memory 1006, or at a later time by storing them and then accessing them from the storage device/s 1010.

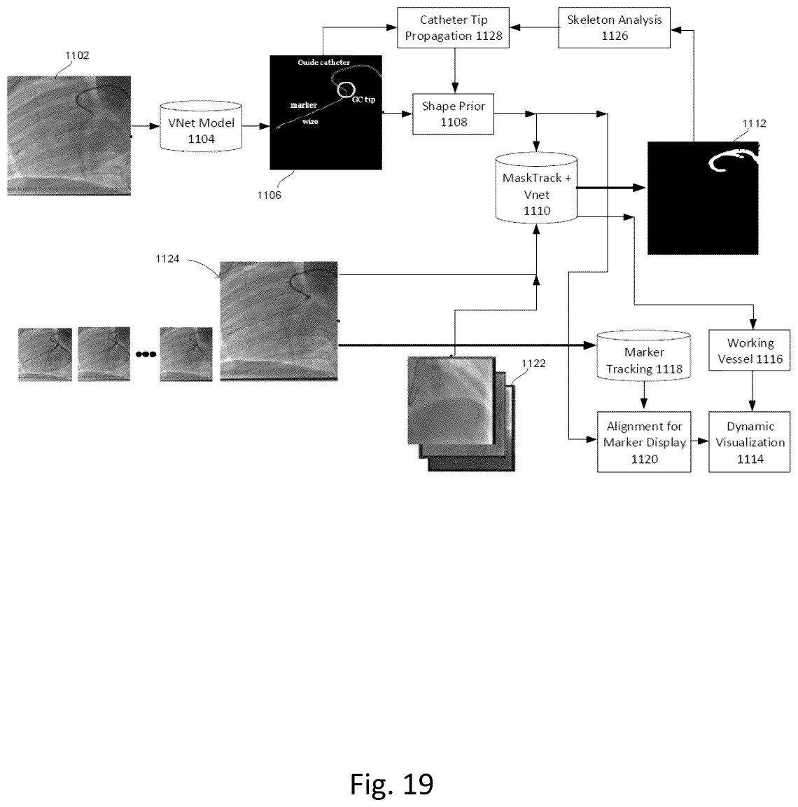

FIG. 19 illustrates an example flow chart of a method of tracking a device through the vasculature according to an aspect of the disclosure. The following operations do not have to be performed in the precise order described below. Rather, various operations can be handled in a different order or simultaneously, and operations may be added or omitted.

To identify the working vessel, an extraluminal image frame 1102 may be received. In some examples, the image frame 1102 may be a fluoroscopic image, captured with or without contrast within the vessel. The extraluminal image frame 1102 may be captured prior to the delivery of the device. For example, the image frame 1102 may be captured after the guidewire and/or catheter (e.g. pigtail catheter) is inserted but before the device, e.g., prosthetic heart valve, is delivered.

The catheter (e.g. pigtail catheter) and/or guidewire within the image frame 1102 may be segmented using AI. The AI may include a deep learning model, such as VNet model 1104. The VNet model 1104 may be trained to predict a plurality of classes, such as catheter, guidewire, device marker, and background. The VNet model 1104 may receive, as input, the first image frame 1102 and may output multi-label segmentation.

The multi-label segmentation output by the VNet model may be converted into a multi-label mask 1106. The multi-label mask 1106 may illustrate the working vessel region as a shape prior to the subsequent process. According to some examples, a fluoroscopic image captured without contrast in the vessel, e.g., a pre-contrast frame, may be used to create a multi-label mask or binary mask. The binary mask created using the pre-contrast frame may be used as a shape prior for the post-contrast frame. Post-contrast frames may be extraluminal image frames captured after contrast has been injected such that the extraluminal image frames include at least some contrast. According to some examples, after contrast is injected and extraluminal images of the vessel are captured, the contrast may highlight the working vessel as well as side branches. The contrast may, in some examples, make it more challenging to detect the guidewire in the extraluminal images. Therefore, using the shape prior of the guidewire and/or catheter obtained from the pre-contrast frame, may allow for the working vessels in post-contrast extraluminal images to be segmented.

According to some examples, a pre-contrast frame may include a guidewire and/or a catheter (e.g. a pigtail catheter). The pre-contrast frame may be processed such that a binary mask is generated. The binary mask may be of the guidewire in the pre-contrast frame. According to some examples, the binary mask may be used to identify guidewire in the working vessel in the post-contrast frame. For example, the binary mask of the guidewire may be overlaid, superimposed, or combined with the post-contrast frame. The shape prior of the guidewire, also referred to as the working wire, may be used to segment the working vessel in post-contrast images.

The shape prior 1108 may be provided as input to a deep learning model 1110. The deep learning model 1110 may be, for example, a MaskTrack+Vnet model. The MaskTrack+Vnet model may be trained to identify the working vessel. The MaskTrack+Vnet model 1110 may be a spatial temporal model. According to some examples, the MaskTrack+Vnet model 1110 may be a spatial-temporal encoder decoder segmentation network. The MaskTrack+Vnet model 1110 may be trained to predict the working vessel.

The MaskTrack+Vnet model 1110 may be trained with a plurality of annotations. A pre-contrast fluoroscopic image may be annotated. The annotations for training the MaskTrack+Vnet model 1110 may be a set of line strips that follow the trajectory of the catheter and guidewire in extraluminal images, e.g., fluoroscopic images, before contrast is injected. The annotations may, in some examples, correspond to the working vessel trajectory. For extraluminal images captured after contrast is injected, the path following the working vessel line may be labeled. For example, post-contrast images may be annotated. The annotations on the post-contrast images may correspond to the work vessel.

When executing the MaskTrack+Vnet model 1110, the input into the MaskTrack+Vnet model 1110 may be an image frame and prediction information from one or more previous frames. For example, if the current frame is frame “t”, the input into the MaskTrack+Vnet model 1110 may be frame “t” and the prediction information from previous frames “t−n”, “t−n+1”, . . . “t−1”, when “n” is greater than 1. According to some examples, the shape prior 1108 of the previous frame may be an additional input into the MaskTrack+Vnet model 1110. The shape prior propagation may correspond to the “MaskTrack” of the MaskTrack+Vnet model 1110. The “VNet” of the MaskTrack+Vnet model 1110 is one type of encoder-decoder segmentation network. However, other segmentation networks may be used as part of the shape prior propagation workflow.

Using the shape prior 1108 may allow for the MaskTrack+Vnet model 1110 to learn, or predict, the anatomical context of the working vessel with respect to the whole vessel tree. According to some examples, the MaskTrack+Vnet model 1110, e.g., the spatial-temporal segmentation model, may not be limited by the previous frame. Rather, in some examples, the shape prior 1108 may be formulated as previous consecutive frames based on the data, or images, to be processed. The MaskTrack+Vnet model 1110 may be executed with a plurality of subsequent frames 1124 being used as input in addition to first frame 1102. The subsequent frames 1124 may be frames captured after the first frame 1102 was captured. In some examples, the subsequent frames 1124 may include contrast. For example, the subsequent frames 1124 may be any combination of fluoroscopic images captured with or without contrast. Based on all the frames, e.g., first frame 1102 and the subsequent frames 1124, the output of the MaskTrack+Vnet model 1110 may be a roadmap of the working vessel 1116.

According to some examples, the catheter and wire tip may be segmented from the subsequent frames 1124. According to some examples, the catheter and wire tip may be segmented from the subsequent frames using the MaskTrack+Vnet model 1110. In a typical workflow, the subsequent images frames 1124 are not recorded. The number of recorded subsequent frames 1124, such as fluoroscopic images captured with a low dose of contrast, is typically less than the number of fluoroscopic images captured with a high-dose of contrast taken during the diagnostic phase. To address the reduced number of fluoroscopic images captured with a low dose of contrast typically captured, the system may augment the fluoroscopic images captured with a high dose of contrast to generate augmented fluoroscopic images captured with a low dose of contrast 1122 such that the augmented fluoroscopic images 1122 can be used as input into the training process for the MaskTrack+Vnet model 1110 in the example of catheter and wire tip segmentation.

According to some examples, at least two factors may be applied to the contrast and signal-noise-ration (“SNR”) to transform fluoroscopic images captured with a high dose contrast into augmented fluoroscopic images 1122 that can be used to train the MaskTrack+Vnet model 1110, or a similar model, to segment the catheter and wire tip. The fluoroscopic images captured with a high dose of contrast captured before delivery of a device, e.g., a prosthetic heart valve, or another workflow, may be processed to extract the neighborhood of target objects, such as the catheter, wire tip, or the like. In some examples, the fluoroscopic images captured with a high dose of contrast may be processed using dilation to extract the neighborhood of target objects. The intensity contrast of the target objects in the fluoroscopic images captured with a high dose of contrast may be determined. In some examples, a signal-noise-ration (“SNR”) may be determined based on the fluoroscopic images captured with a high dose of contrast and the subsequent frames of fluoroscopic images captured with a low dose of contrast 1124. According to some examples, the augmented fluoroscopic images 1122 may be generated using generative adversarial network (“GAN”) type models, or other generative type models.

The MaskTrack+Vnet model 1110 may be trained using the augmented fluoroscopic images 1122 as well as fluoroscopic images captured with a low dose of contrast, such as subsequent frames 1124, as input. The MaskTrack+Vnet model 1110 may be trained to automatically detect the catheter and wire tip. The MaskTrack+Vnet model 1110 may be trained to predict a segmentation of the catheter and wire tip. For example, when executed, the MaskTrack+Vnet model 1110 may receive subsequent frames 1124 and provide frame 1112 as output, where frame 1112 may be the segmented catheter and/or the wire tip.

In some examples, frame 1112 may be a skeleton frame. The skeleton frame 1112 may be analyzed 1126 for reference point propagation. For example, the skeleton frame may contain one or more side branches, other than the working vessel. According to some examples, to keep the shape prior substantially similar to the working vessel, skeleton analysis 1126 may be performed. Skeleton analysis 1126 may include, for example, skeleton path search, side path removal, etc. In some examples, the output from MaskTrack+Vnet 1110 is a working vessel map that is obtained after injecting contrast into the vessel. The catheter tip may be invisible due to the occlusion by contrast X-ray attenuation. The shape prior from frame 1106 showing the multi-labels can be aligned with the skeleton analysis 1126 result using a geometric transformation. After the alignment, the catheter tip in frame 1112 may be projected to the nearest neighboring skeleton point as the propagated catheter tip 1128 in the fluoroscopic images captured with contrast injected into the vessel.

According to some examples, the frames 1124 may be used to detect and track 1118 the position of the marker on the device being delivered within the vessel. The markers on the device may be detected and tracked 1118 using a machine learning model, a convolution neural network, or the like.

The working vessel map 1116 along with the catheter tip location may be obtained using MaskTrack+Vnet 1110 for the fluoroscopic images captured with a high dose of contrast. For example, the catheter tip location may be automatically identified by MaskTrack+Vnet 1110 along where a curve going through the main vessel. With a similar approach, the catheter and its tip location can be identified for the live fluoroscopic images. The device markers in the fluoroscopic images captured during the procedure, e.g., live fluoroscopic images, may be detected and tracked. In some examples, the imaging posture between roadmap and fluoroscopic images captured during the procedure may be slightly different. In such an example, the image context between them may not be aligned. To address this issue, the catheter tip in both the roadmap and the fluoroscopic images captured during the procedure can be aligned by translating one image frame to another, so that the image context can be properly fused together. The markers can then be displayed on the working vessel using nearest neighboring points or other geometric projection for marker display 1120.

The working vessel 1116 may be provided for output as a roadmap when dynamically visualizing 1114 the delivery of the device, e.g., a prosthetic heart valve, in the vessel. The detected and tracked markers 1118, once aligned 1120, may be provided for output. For example, the markers may be provided for output on the vessel map to provide an indication of the location of the device in real time, without having to inject additional contrast. According to some examples, a treatment zone, initially identified on the vessel map, may be provided for display on the fluoroscopic images captured during the procedure such that the location of the device on the fluoroscopic images can be seen relative to the target 500. In some examples, the marker may be provided for output on the roadmap to provide an indication of the location of the device in real time with respect to the target 500.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of implanting a medical device into a location within a heart of a patient, the location being within a target site of the patient, the method comprising:
generating a series of baseline fluoroscopic images of the target site of the patient while contrast media is within the target site, the series of baseline fluoroscopic images encompassing at least one complete heartbeat cycle of a heart of the patient;
annotating the series of baseline fluoroscopic images to provide at least one of (i) an anatomical landmark annotation representing an anatomical landmark of the patient, or (ii) a target annotation representing a target location for deploying the medical device;
advancing the medical device into the patient toward the target site while the medical device is mounted to or in a delivery device in a collapsed condition;
generating real-time fluoroscopic images of the target site while the medical device is located within the target site;
displaying the real-time fluoroscopic images on a display device such that the (i) anatomical landmark annotation and/or the (ii) target annotation from the series of baseline fluoroscopic images is overlaid on the display of the real-time fluoroscopic images; and
deploying the medical device into the location.

2. The method of claim 1, wherein the medical device is a prosthetic heart valve, and the location is a heart valve of the patient.

3. The method of claim 2, wherein annotating the series of baseline fluoroscopic images includes providing both (i) the anatomical landmark annotation representing the anatomical landmark of the patient and (ii) the target annotation representing the target location for deploying the prosthetic heart valve.

4. The method of claim 2, wherein annotating the series of baseline fluoroscopic images includes annotating each image in the series so that every image that encompasses at least one compete heartbeat cycle of the heart of the patient includes the annotation.

5. The method of claim 2, further comprising co-registering the series of baseline fluoroscopic images to the real-time fluoroscopic images so that each image in the series of baseline images is registered to a corresponding real-time fluoroscopic image.

6. The method of claim 5, wherein the co-registration is performed so that each of the real-time fluoroscopic images that represents a given point within the complete heartbeat cycle of the heart corresponds to an image in the series of the baseline fluoroscopic images that represents the given point within the complete heartbeat cycle.

7. The method of claim 2, wherein annotating the series of baseline fluoroscopic images includes providing the anatomical landmark annotation representing the anatomical landmark of the patient, the anatomical landmark being a plane of an annulus of the heart valve.

8. The method of claim 2, wherein annotating the series of baseline fluoroscopic images includes providing the target annotation representing the target location for deploying the prosthetic heart valve, the target location being a set distance from a plane of an annulus of the heart valve.

9. The method of claim 8, wherein the set distance is non-zero.

10. The method of claim 8, wherein the set distance results in the target annotation being positioned on an inflow side of the heart valve.

11. The method of claim 10, wherein the heart valve is a native aortic valve, the prosthetic heart valve is a prosthetic aortic valve, and the target annotation is positioned in a ventricular side of the native aortic valve.

12. The method of claim 11, wherein prior to deploying the prosthetic aortic valve into the native aortic valve, an inflow end of the prosthetic aortic valve is aligned with the target annotation overlaid on the display of the real-time fluoroscopic images.

13. The method of claim 8, wherein the set distance is based on (i) device-specific information relating to a device parameter of the prosthetic heart valve and/or (ii) patient-specific information relating to an anatomical parameter of an anatomy of the patient.

14. The method of claim 1, wherein during the generation of the series of baseline fluoroscopic images of the target site of the patient, at least a portion of an accessory wire is located within the target site.

15. The method of claim 14, wherein the accessory wire includes a plurality of radiopaque markers, each adjacent pair of radiopaque markers spaced apart from each other along the accessory wire at a known distance.

16. The method of claim 15, further comprising annotating the series of baseline fluoroscopic images to draw a line between two of the radiopaque markers on the display device to correlate the known distance to a pixel size on the display device.

17. The method of claim 1, wherein generating the series of baseline fluoroscopic images of the target site of the patient is performed with a static fluoroscopic imager.

18. The method of claim 2, wherein generating the series of baseline fluoroscopic images of the target site of the patient is performed with a dynamic fluoroscopic imager that sweeps around a point to generate the fluoroscopic images along different imaging planes.

19. The method of claim 18, wherein the heart valve is a native aortic valve, the prosthetic heart valve is a prosthetic aortic valve, and the point is a radial center of a native annulus of the native aortic valve.

20. The method of claim 1, further comprising generating audible and/or tactile feedback as the prosthetic heart valve moves closer (i) the anatomical landmark annotation or (ii) the target annotation overlaid on the display of the real-time fluoroscopic images.

21. The method of claim 1, further comprising generating visual feedback as the prosthetic heart valve moves closer (i) the anatomical landmark annotation or (ii) the target annotation overlaid on the display of the real-time fluoroscopic image, wherein the visual feedback including a change in a displayed color of (i) the anatomical landmark annotation or (ii) the target annotation overlaid on the display of the real-time fluoroscopic image.

22. The method of claim 1, wherein the series of baseline fluoroscopic images is generated at a first resolution, and the real-time fluoroscopic images are generated at a second resolution that is different from the first resolution.

23. The method of claim 22, wherein the first resolution is higher than the second resolution.

24. The method of claim 2, wherein the series of baseline fluoroscopic images of the target site of the patient are generated while the prosthetic heart valve is at the target site.

25. The method of claim 1, wherein the medical device is a collapsible and expandable left atrial appendage occluder, and the location is a left atrial appendage of the patient.
